(19) United States
(12) Patent Application Publication
(10) Pub. No.: US 2024/0043895 A1
(43) Pub. Date: Feb. 8, 2024

(54) SPRAY DRIED EXTRACT

(71) Applicant: Sutro Biopharma, Inc., South San Francisco, CA (US)

(72) Inventors: Douglas Lee Gettel, Walnut Creek, CA (US); James Zawada, Redwood City, CA (US); Robert David Kiss, San Mateo, CA (US); Allison Dwyer, Burlingame, CA (US); Amarjeet Singh, Suisun City, CA (US); Richard Alan Johnson, Nottingham (GB); Lisa Sawicki Pieri, Somerville, MA (US); Dara Patrick Dowlatshahi, Menlo Park, CA (US)

(21) Appl. No.: 18/231,204

(22) Filed: Aug. 7, 2023

Related U.S. Application Data
(60) Provisional application No. 63/396,143, filed on Aug. 8, 2022.

(51) Int. Cl. C12P 21/00; C12N 1/04; C12N 1/06; C12N 15/70

(52) U.S. Cl. CPC C12P 21/00; C12N 1/04; C12N 1/06; C12N 15/70; C12N 2500/34

(57) ABSTRACT

This disclosure provides for an improved method of stabilizing spray-dried bacterial extracts with a composition comprising a stabilizer such that the extracts can be used in cell-free protein synthesis. Also provided herein are formulations for stable, spray-dried bacterial extracts that have increased protein synthesis activity compared to spray-dried bacterial extracts that do not contain a stabilizer.

### CROSS-REFERENCE TO RELATED APPLICATION

This application claims priority to U.S. Provisional Application No. 63/396,143, filed Aug. 8, 2022, the disclosure of which is herein incorporated by reference in its entirety for all purposes.

### BACKGROUND OF THE INVENTION

The present disclosure provides compositions and methods for producing spray-dried bacterial extracts having enhanced stability after long term storage at different temperatures. The spray-dried extracts can be used in commercial scale cell-free protein synthesis reactions.

### BRIEF SUMMARY OF THE INVENTION

Provided herein are compositions and methods for making spray-dried bacterial extracts for use in cell-free protein synthesis reactions. In one aspect, the method comprises: i) combining a bacterial extract comprising lysed bacterial components with a composition comprising trehalose, lactose, leucine, or raffinose to yield a mixture, wherein the bacterial extract is able to synthesize a target protein from a template nucleic acid encoding the target protein in cell-free protein synthesis reaction; and ii) spray-drying the mixture to produce the stable, spray-dried bacterial extract.

In another aspect, the disclosure provides a method for expressing a target protein in a cell-free protein synthesis reaction, the method comprising: i) combining a bacterial extract comprising lysed bacterial components with a composition comprising trehalose, lactose, leucine, or raffinose to yield a mixture, wherein the lysed bacterial components can synthesize a target protein from a template nucleic acid encoding the target protein in cell-free protein synthesis reaction; ii) spray-drying the mixture to produce the stable, spray-dried bacterial extract, iii) rehydrating the spray-dried bacterial extract; iv) adding a template nucleic acid encoding the target protein to the rehydrated extract, wherein the template nucleic acid is translated in the rehydrated extract, thereby expressing the target protein.

In another aspect, the disclosure provides a method for expressing a target protein in a cell-free protein synthesis reaction, the method comprising: rehydrating a spray-dried bacterial extract comprising lysed bacterial components and a composition comprising trehalose, lactose, leucine, or raffinose; adding a template nucleic acid encoding the target protein to the rehydrated extract, wherein the template nucleic acid is translated in the rehydrated extract, thereby expressing the target protein.

In some embodiments, the bacterial extract comprising lysed bacterial components is a liquid or rehydrated bacterial extract.

In some embodiments, the composition comprises trehalose or lactose. In some embodiments, the mixture comprises about 25 to 200 g/kg trehalose. In some embodiments, the mixture comprises about 50 to 100 g/kg trehalose. In some embodiments, the trehalose is trehalose dihydrate (TDH).

In some embodiments, the mixture comprises about 25 to 200 g/kg lactose. In some embodiments, the mixture comprises about 50 to 100 g/kg lactose. In some embodiments, the lactose is lactose monohydrate (LMH).

In some embodiments, the mixture comprises about 5 to 10 g/L of leucine. In some embodiments, the mixture comprises about 25 to 200 g/L raffinose.

In some embodiments, the stable, spray-dried bacterial extract comprises about 40 to 70 g/L of the bacterial extract solids. In some embodiments, the stable, spray-dried bacterial extract comprises about 50 to 60 g/L of the bacterial extract solids.

In some embodiments, the bacterial extract is further combined with one or more of high glass transition temperature (Tg), nonpolar, uncharged amino acids. In some embodiments, the one or more high Tg nonpolar, uncharged amino acids are selected from Valine, Tryptophan, Isoleucine, Leucine, Alanine, Glycine, Proline, and any combinations thereof. In some embodiments, the one or more high Tg nonpolar, uncharged amino acids are selected from L-Valine, L-Tryptophan, L-Isoleucine, L-Leucine, L-Alanine, Glycine, or L-Proline, and any combinations thereof.

In some embodiments, the bacterial extract is further combined with one or more amino acids selected from leucine, glycine, alanine, valine, isoleucine, proline, tryptophan, serine, threonine, methionine, asparagine, glutamine, cysteine, aspartic acid, glutamic acid, histidine, lysine, arginine, and any combinations thereof.

In some embodiments, the mixture comprises 5 to 15 g/kg of amino acids.

In some embodiments, in step (i), the mixture further comprises maltodextrin, sucrose, mannitol, sorbitol, polyethylene glycol 200, polysorbate 80 (Tween® 80), polyvinylpyrrolidone (PVP or Kollidon 12 PF), or 2-Hydroxypropyl-β-Cyclodextrin.

In some embodiments, the bacterial spray-dried extract comprises less than or equal to about 15% (w/w) residual water. In some embodiments, the bacterial spray-dried extract comprises less than or equal to about 10% (w/w) residual water. In some embodiments, the bacterial spray-dried extract comprises less than or equal to about 5% (w/w) residual water.

In some embodiments, the bacterial spray-dried extract is able to synthesize the target protein with a titer of at least 80% relative to a control extract after storage at 2° C. to 8° C. for at least 6 months. In some embodiments, the bacterial spray-dried extract is able to synthesize the target protein with a titer of at least 80% relative to a control extract after storage at 2° C. to 8° C. for at least 12 months. In some embodiments, the bacterial spray-dried extract is able to synthesize the target protein with a titer of at least 80% relative to a control extract after storage at 2° C. to 8° C. for at least 18 months.

In some embodiments, the bacterial spray-dried extract is able to synthesize the target protein with a titer of at least 80% relative to a control extract after storage at about −20° C. for at least 6 months. In some embodiments, the bacterial spray-dried extract is able to synthesize the target protein with a titer of at least 80% relative to a control extract after storage at about −20° C. for at least 12 months. In some embodiments, the bacterial spray-dried extract is able to synthesize the target protein with a titer of at least 80% relative to a control extract after storage at about −20° C. for at least 18 months.

In some embodiments, the bacterial spray-dried extract is able to synthesize the target protein with a titer of at least 80% relative to a control extract after storage at about room temperature (20° C.) for at least 6 months. In some embodiments, the bacterial spray-dried extract is able to synthesize the target protein with a titer of at least 80% relative to a control extract after storage at about room temperature (20° C.) for at least 12 months. In some embodiments, the bacterial spray-dried extract is able to synthesize the target protein with a titer of at least 80% relative to a control extract after storage at about room temperature (20° C.) for at least 18 months.

In some embodiments, the titer is determined by the Phytip® method.

In some embodiments, the spray-dried extract and the control extract comprise trehalose, and the spray-dried extract is stored at 2° C. to 8° C. for at least 12 months and following reconstitution is able to synthesize the target protein with a titer of at least 80% relative to the control extract reconstituted at time zero. In some embodiments, the protein synthesis activity of the spray-dried extract decreases less than about 5% per month compared to a control extract when the spray-dried extract is stored at 2° C. to 8° C. and is then rehydrated.

In some embodiments, the control extract does not comprise trehalose, lactose, leucine, or raffinose. In some embodiments, the control extract comprises trehalose, lactose, leucine, or raffinose.

In some embodiments, the spray-dried bacterial extract comprises an active oxidative phosphorylation system in cell-free protein synthesis. In some embodiments, the bacterial extract is from an Escherichia species.

In some embodiments, prior to step (i), the bacterial extract is heated at about 20° C. to 45° C. for about 30 minutes to about 10 hours.

In some embodiments, the spray-drying in step (ii) comprises atomizing the mixture to produce droplets; contacting the droplets with a gas to evaporate liquid from the droplets; separating the dried extract from the gas and smaller particles; and collecting the spray-dried extract.

In some embodiments, greater than or equal to 90% (w/w) of the liquid is removed from the mixture. In some embodiments, greater than or equal to 95% (w/w) of the liquid is removed from the mixture.

In some embodiments, the method further comprises (iii) rehydrating the spray-dried bacterial extract; and (iv) synthesizing the target protein under conditions that support a cell-free protein synthesis reaction. In some embodiments, the rehydrated bacterial extract comprises about 20% to 60% (by volume) of the cell-free protein synthesis reaction. In some embodiments, the rehydrated bacterial extract comprises about 30% to 40% (by volume) of the cell-free protein synthesis reaction.

In another aspect, the disclosure provides a spray-dried bacterial extract composition for cell-free protein synthesis, the extract comprising dried, lysed bacterial components; and a composition comprising trehalose, lactose, leucine, or raffinose, wherein the extract is able to synthesize upon rehydration a target protein from a template nucleic acid encoding the target protein.

In some embodiments, the composition comprises trehalose or lactose. In some embodiments, the composition comprises about 25 to 200 g/kg of trehalose. In some embodiments, the composition comprises about 50 to 100 g/kg of trehalose. In some embodiments, the trehalose is trehalose dihydrate (TDH). In some embodiments, the composition comprises about 25 to 200 g/kg of lactose. In some embodiments, the composition comprises about 50 to 100 g/kg of lactose. In some embodiments, the lactose is lactose monohydrate (LMH). In some embodiments, the composition comprises about 5 to 10 g/L of leucine. In some embodiments, the composition comprises about 25 to 200 g/L raffinose.

In some embodiments, the extract further comprises one or more high glass transition temperate (Tg) nonpolar uncharged amino acids. In some embodiments, the high Tg nonpolar, uncharged amino acids are selected from Valine, Tryptophan, Isoleucine, Leucine, Alanine, Glycine, Proline, and any combinations thereof. In some embodiments, the high Tg nonpolar, uncharged amino acids are selected from the group consisting of L-Valine, L-Tryptophan, L-Isoleucine, L-Leucine, L-Alanine, Glycine, L-Proline, and any combinations thereof. In some embodiments, the extract comprises 5 to 15 g/kg of amino acids.

In some embodiments, the extract further comprises one or more, a combination of all, or a subset of amino acids selected from leucine, glycine, alanine, valine, isoleucine, proline, tryptophan, serine, threonine, methionine, asparagine, glutamine, cysteine, aspartic acid, glutamic acid, histidine, lysine, and arginine. In some embodiments, the extract comprises about 5 to 15 g/L of the amino acids.

In some embodiments, the extract further comprises maltodextrin, sucrose, mannitol, sorbitol, polyethylene glycol 200, polysorbate 80 (Tween® 80), polyvinylpyrrolidone (PVP or Kollidon 12 PF), or 2-Hydroxypropyl-β-Cyclodextrin.

In some embodiments, the spray-dried extract comprises less than or equal to about 15% (w/w) residual water. In some embodiments, the spray-dried extract comprises less than or equal to about 10% (w/w) residual water. In some embodiments, the spray-dried extract comprises less than or equal to about 5% (w/w) residual water.

In some embodiments, the spray-dried extract is stored at 2° C. to 8° C. for at least 6 months and is able to synthesize the target protein with a titer of at least 80% relative to a control extract. In some embodiments, the spray-dried extract is stored at 2° C. to 8° C. for at least 12 months and is able to synthesize the target protein with a titer of at least 80% relative to a control extract. In some embodiments, the spray-dried extract is stored at 2° C. to 8° C. for at least 18 months and is able to synthesize the target protein with a titer of at least 80% relative to a control extract.

In some embodiments, the spray-dried extract is stored at about −20° C. for at least 6 months and is able to synthesize the target protein with a titer of at least 80% relative to a control extract. In some embodiments, the spray-dried extract is stored at about −20° C. for at least 12 months and is able to synthesize the target protein with a titer of at least 80% relative to a control extract. In some embodiments, the spray-dried extract is stored at about −20° C. for at least 18 months and is able to synthesize the target protein with a titer of at least 80% relative to a control extract.

In some embodiments, the spray-dried extract is stored at about room temperature (20° C.) for at least 6 months and is able to synthesize the target protein with a titer of at least 80% relative to a control extract. In some embodiments, the spray-dried extract is stored at about room temperature (20° C.) for at least 12 months and is able to synthesize the target protein with a titer of at least 80% relative to a control extract. In some embodiments, the spray-dried extract is stored at about room temperature (20° C.) for at least 18 months and is able to synthesize the target protein with a titer of at least 80% relative to a control extract.

In some embodiments, the titer is determined by the Phytip® method. In some embodiments, the control extract does not comprise trehalose, lactose, leucine, or raffinose. In some embodiments, the control extract comprises trehalose, lactose, leucine, or raffinose.

In some embodiments, the spray-dried extract and the control extract comprise trehalose, and the spray-dried extract is stored at 2° C. to 8° C. for at least 12 months and following rehydration is able to synthesize the target protein with a titer of at least 80% relative to the control extract rehydrated at time T=zero.

In some embodiments, the protein synthesis activity of the rehydrated spray-dried extract decreases at less than about 5% per month when the extracts are stored at 2° C. to 8° C. prior to rehydration. In some embodiments, the protein synthesis activity of the rehydrated spray-dried extract is greater than or equal to the protein synthesis activity of rehydrated control spray-dried extract when the extracts are stored at 2° C. to 8° C. for greater than or equal to 8 months prior to rehydration. In some embodiments, the spray-dried extracts are stored at 2° C. to 8° C. for 13 months prior to rehydration. In some embodiments, the control spray-dried extract does not comprise trehalose.

In some embodiments, the spray-dried extract comprises trehalose or lactose, is stored at 2° C. to 8° C. for at least 4 months and is able to synthesize the target protein with a titer of at least 75% relative to a control extract stored at −20° C. that does not comprise trehalose or lactose. In some embodiments, the spray-dried extract comprises about 75 g/kg to 105 g/kg trehalose or about 100 g/kg lactose.

In some embodiments, the rehydrated extract has greater than or equal to 80% of the initial protein synthesis activity when compared to the protein synthesis activity of rehydrated extract at T=0 when stored at 2° C. to 8° C. for at least 18 months prior to rehydration.

In some embodiments, the spray-dried bacterial extract has an active oxidative phosphorylation system in cell-free protein synthesis. In some embodiments, the extract is from an Escherichia species. In some embodiments, the extract is a powder. In some embodiments, the extract does not have a cake-like appearance or is not a dried cake.

In another aspect, the disclosure provides a method of preparing a spray-dried extract, comprising the steps of: (i) providing a liquid bacterial extract comprising components for cell-free synthesis of a target protein from a template nucleic acid encoding the target protein; (ii) producing droplets of the liquid bacterial extract; (iii) contacting the droplets with a gas to evaporate liquid from the droplets; (iv) separating the dried extract from the gas and smaller particles; and (v) collecting the spray-dried extract.

In some embodiments, prior to step (i) the liquid bacterial extract is sterile filtered. In some embodiments, the sterile filtered liquid bacterial extract is activated by heating.

In some embodiments, a composition comprising trehalose, lactose, leucine, or raffinose is added to the activated sterile filtered liquid bacterial extract prior to step (ii). In some embodiments, the composition comprises about 25 to 200 g/kg trehalose, about 25 to 200 g/kg lactose, about 5 to 10 g/L leucine, or about 25 to 200 g/L raffinose.

In some embodiments, step (i) further comprises adding one or more amino acids to the activated sterile filtered liquid bacterial extract. In some embodiments, the one or more amino acids comprise high glass transition temperate (Tg) nonpolar uncharged amino acids selected from the group consisting of L-Valine, L-Tryptophan, L-Isoleucine, L-Leucine, L-Alanine, Glycine, L-Proline, and any combinations thereof. In some embodiments, the one or more amino acids are selected from leucine, glycine, alanine, valine, isoleucine, proline, tryptophan, serine, threonine, methionine, asparagine, glutamine, cysteine, aspartic acid, glutamic acid, histidine, lysine, and arginine.

In some embodiments, one or more of maltodextrin, sucrose, mannitol, sorbitol, polyethylene glycol 200, polysorbate 80 (Tween® 80), polyvinylpyrrolidone (PVP or Kollidon 12 PF), 2-Hydroxypropyl-β-Cyclodextrin, or any combinations thereof, are added to the activated sterile filtered liquid bacterial extract.

In some embodiments, step (ii) comprises atomizing the liquid bacterial extract to produce the droplets. In some embodiments, the atomizing comprises passing the liquid bacterial extract through an atomization device selected from a nozzle or rotary atomizer. In some embodiments, the median droplet size is from about 20 to 100 microns (Dv50) at an atomized gas pressure of 10 to 50 psig.

In some embodiments, step (iii) comprises contacting the droplets with a drying gas through a drying chamber, wherein the drying gas has an outlet temperature of about 60° C. to about 90° C. In some embodiments, step (iv) comprises separating the dried extract from the gas and smaller particles using centrifugal force. In some embodiments, step (v) comprises collecting the spray-dried extract in a container.

In some embodiments, the collected spray-dried extract comprises less than or equal to about 15%, 10% or 5% (w/w) residual water. In some embodiments, greater than or equal to 85%, 90%, or 95% (w/w) of the liquid is removed from the spray-dried extract.

In some embodiments, the protein synthesis activity of the rehydrated spray-dried extract decreases less than about 5% per month when the extracts are stored at 2° C. to 8° C. prior to rehydration. In some embodiments, the protein synthesis activity of the rehydrated spray-dried extract is greater than or equal to the protein synthesis activity of rehydrated control extract when the extracts are stored at 2° C. to 8° C. for greater than or equal to 8 months prior to rehydration. In some embodiments, the spray-dried extracts are stored at 2° C. to 8° C. for 13 months prior to rehydration.

In some embodiments, the spray-dried bacterial extract has an active oxidative phosphorylation system in cell-free protein synthesis. In some embodiments, the liquid extract is from an Escherichia species.

In another aspect, the disclosure provides a spray-dried extract for cell-free protein synthesis, the spray dried extract comprising dried, lysed bacterial components and one or more of a stabilizer, wherein the stabilizer has a glass transition temperature (Tg) of at least about 90° C., and wherein the concentration of the stabilizer is between about 5 g/L and 200 g/L or between about 25 g/kg and 200 g/kg in the liquid extract prior to spray-drying.

In some embodiments, the stabilizer is selected from trehalose, lactose, and leucine. In some embodiments, the stabilizer comprises about 25 to 200 g/kg trehalose, about 25 to 200 g/kg lactose, or about 5 to 10 g/L of leucine. In some embodiments, the trehalose is trehalose dihydrate (TDH) and the lactose is lactose monohydrate (LMH). In some embodiments, greater than or equal to 85%, 90% or 95% (w/w) of the liquid is removed from the mixture.

In some embodiments, the extract further comprises one or more high glass transition temperate (Tg) nonpolar uncharged amino acids selected from L-Valine, L-Tryptophan, L-Isoleucine, L-Leucine, L-Alanine, Glycine, L-Proline, and any combinations thereof. In some embodiments, the extract further comprises one or more, a combination of all, or a subset of amino acids selected from leucine, glycine, alanine, valine, isoleucine, proline, tryptophan, serine, threonine, methionine, asparagine, glutamine, cysteine, aspartic acid, glutamic acid, histidine, lysine, and arginine. In some embodiments, the extract further comprises maltodextrin, sucrose, mannitol, sorbitol, polyethylene glycol 200, polysorbate 80 (Tween® 80), polyvinylpyrrolidone (PVP or Kollidon 12 PF), 2-Hydroxypropyl-β-Cyclodextrin or any combinations thereof.

In another aspect, provided is a method for producing a target protein from a spray dried extract, the method comprising: reconstituting a spray dried extract of the disclosure; providing a template nucleic acid encoding the target protein; and generating the target protein.

### BRIEF DESCRIPTION OF THE DRAWINGS

FIG. 1 shows data from laboratory-scale spray drying. Drying was performed on a lab scale Buchi B-290 spray dryer with respective inlet and outlet temperatures noted in parenthesis for dried samples as (Inlet Temperature/Outlet Temperature). XpressCF® (XCF) testing used batch reactions in a Micro-24 microbioreactor (m24) and Flower Plate (FP) with 30% (by volume) XtractCF® to express product anti-CD74 antibody. Source liquid and dried XtractCF® was from lot DR 3c H4 B5.

FIG. 2A shows data from a pilot-scale spray drying formulation and drying process. Samples were prepared and dried formulated with 100 g/L, 50 g/L, and 25 g/L trehalose. Drying was performed on a pilot scale Mobile Minor PSD-1 spray dryer. XCF testing used batch reactions in a Micro-24 microbioreactor with 30% (by volume) XtractCF® to express product anti-CD74 antibody. Source liquid and dried XtractCF® was from lot ER 15-4.

FIG. 2B shows data from a pilot-scale spray drying formulation and drying process. Samples were prepared and dried formulated with 100 g/L, 50 g/L, and 25 g/L trehalose. Drying was performed on a pilot scale Mobile Minor PSD-1 spray dryer. XCF testing used batch reactions in a Micro-24 microbioreactor with 30% (by volume) XtractCF® to express product anti-CD74 antibody. Source liquid and dried XtractCF® was from lot ER 15-4.

FIG. 3A, FIG. 3B, and FIG. 3C show long term stability data of spray dried trehalose dihydrate formulated XtractCF® at 2-8° C., −20° C., and room temperature, respectively. Samples were spray dried on a Mobile Minor (PSD-1) with trehalose dihydrate formulation at 25, 50, and 100 g/L amounts and associated residual moisture levels (batch and % residual moisture noted in the legend). The Rates of Titer Loss (%/month) were calculated from linear fits of data from 6 to 24.5 months. Titer of antibody product anti-CD74 antibody was measured for the initial time point (t₀) and trastuzumab antibody was used for all other times in this study.

FIG. 4A shows data from 100 L demonstration runs from a 100 L spray drying study. Drying was done using a modified PSD-2. XCF testing used batch reactions in a Micro-24 microbioreactor with 30% extract (by volume) to express product trastuzumab. Source liquid and dried XtractCF® was (1) demonstration run (DR) 1, extract lot ER11:ER17 80%:20% (2) DR 2, extract lot ER17 (3) DR 3, extract lot ER 18.

FIG. 4B shows data from demonstration runs from a 100 L spray drying study. Drying was done using a modified PSD-2. XCF testing used batch reactions in a Micro-24 microbioreactor with 30% extract to express product trastuzumab. Source liquid and dried XtractCF® was (1) DR 1, ER11:ER17 80%:20% (2) DR 2, ER17 (3) DR 3, ER 18.

FIG. 5A shows long term stability data at 2-8° C. from a 100 L spray drying study. XCF testing used batch reactions in a Micro-24 microbioreactor with 30% (by volume) extract to express product trastuzumab.

FIG. 5B shows long term stability data at −20° C. from a 100 L spray drying study. XCF testing was using batch reactions in a Micro-24 microbioreactor with 30% (by volume) extract to express product trastuzumab.

FIG. 5C shows long term stability data at room temperature from a 100 L spray drying study. XCF testing was using batch reactions in a Micro-24 microbioreactor with 30% (by volume) extract to express product trastuzumab.

FIG. 6A shows long term stability data at −20° C., 2-8° C., and room temperature from a 100 L spray drying study. XCF testing was using batch reactions in a DASbox stirred tank bioreactor with 37.5% (by volume) extract to express product anti-folate receptor alpha antibody with pre-fabricated light chain (PFLC).

FIG. 6B shows long term stability data at −20° C., 2-8° C., and room temperature from a 100 L spray drying study. XCF testing used batch reactions in a DASbox stirred tank bioreactor with 37.5% (by volume) extract to express product anti-folate receptor alpha antibody with PFLC.

FIG. 7A and 7B show comparative data of different trehalose spray drying formulations from a February 2020 Mobile Minor® spray drying study. FIG. 7A shows a comparison of initial activity for 100 g/L trehalose and 75 g/kg trehalose at the same drying process conditions (Inlet T/Outlet T; 168° C./80° C.). XCF testing used 37.5% (by volume) extract and expressed anti-folate receptor alpha antibody with PFLC. FIG. 7B shows a comparison of initial activity for 100 g/L trehalose and 75 g/kg trehalose at the same drying process conditions (Inlet T/Outlet T; 168° C./80° C.). XCF testing used 37.5% (by volume) extract and expressed anti-CD74 antibody.

FIG. 8A shows comparative data of different drying conditions for lactose spray drying formulations from the February 2020 Mobile Minor® spray drying study. FIG. 8A shows a comparison of initial activity for 100 g/kg lactose for three different drying process conditions (Inlet T/Outlet T; 168° C./80° C., 150° C./70° C., 117° C./60° C.). XCF testing used 37.5% (by volume) extract and expressed anti-folate receptor alpha antibody with PFLC.

FIG. 8B shows comparative data of different drying conditions for lactose spray drying formulations from the February 2020 Mobile Minor® spray drying study. FIG. 9B shows a comparison of initial activity for 100 g/kg lactose for three different drying process conditions (Inlet T/Outlet T; 168° C./80° C., 150° C./70° C., 117° C./60° C.). XCF testing used 37.5% (by volume) extract and expressed anti-CD74 antibody.

FIG. 9 shows long term stability data at 2-8° C. for trehalose dihydrate and lactose monohydrate single component formulated XtractCF®. FIG. 9 shows data from samples spray dried on a Mobile Minor® (PSD-1) with trehalose dihydrate formulation at 75 g/kg and 100 g/L amounts, and lactose monohydrate at 100 g/kg and associated residual moisture levels. The Rates of Titer Loss (%/month) are calculated from linear fits of data from 1 to 5 months. Titer of trastuzumab antibody was measured in this study.

FIG. 10 shows activity data for PSD-3. XCF testing is all in flower plate with 30% extract expressing trastuzumab.

### DETAILED DESCRIPTION

The present disclosure provides compositions and methods for making spray-dried bacterial extracts for use in cell-free protein synthesis reactions. The spray-dried bacterial extracts comprise an additive (e.g., one or more additives) that increases the stability of the extract during long term storage compared to spray-dried extracts that do not comprise the additive. The spray-dried extracts provide the unexpected advantage of retaining their capacity to produce biomolecules, e.g., proteins, when stored at different temperatures over relatively long periods of time compared to spray-dried extracts that do not contain the additive. For example, the spray-dried extracts can unexpectedly be stored at about −20° C. (minus 20° C.), at about 2° C. to 8° C., or at about room/ambient temperature (e.g., about 20° C.) for 6 months to 18 months (or longer) and still retain their capacity to produce biomolecules, e.g., proteins. In contrast, liquid bacterial extracts stored at about −20° C. with or without additives typically retain activity for only a few months, and liquid bacterial extracts stored at about 2° C. to 8° C. without additives typically lose about 25 to 50% activity after one day.

Spray-drying bacterial extracts also provides the advantage of both reducing the volume of the liquid extract and increasing the stability of the bacterial extract for long term storage for commercial scale production.

### I. DEFINITIONS

As used herein, the following terms have the meanings ascribed to them unless specified otherwise.

It is to be understood that this disclosure is not limited to the particular methodology, protocols, cell lines, animal species or genera, and reagents described, as such may vary. It is also to be understood that the terminology used herein is for the purpose of describing particular embodiments only and is not intended to limit the scope of the present disclosure, which will be limited only by the appended claims.

As used herein the singular forms “a,” “and,” and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this disclosure belongs unless clearly indicated otherwise.

In the claims. the transitional term “comprising” is a term of art and is considered to be inclusive or open-ended and does not exclude additional, unrecited elements or method steps. The term “consisting essentially of” refers to the specified materials or steps and those that do not materially affect the basic and novel characteristic(s) of the claimed subject matter. The terms “consists of” or “consisting of” excludes any element, step, or ingredient not specified in the claim.

As used herein, the term “about,” when modifying any amount, refers to the variation in that amount typically encountered by one of skill in the art, e.g., in protein synthesis experiments. For example, the term “about” refers to the normal variation encountered in measurements for a given analytical technique, both within and between batches or samples. Thus, the term about can include variation of 1-10% of the measured value, such as 1%, 2%, 3%, 4%, 5%, 6%, 7%, 8%, 9%, or 10% variation of the measured value. The amounts disclosed herein include equivalents to those amounts, including amounts modified or not modified by the term “about.”

The term “additive,” also referred to as a “stabilizer” or “excipient” refers to a compound or composition that is added to or combined with a liquid bacterial extract prior to spray-drying.

The term “bacterial extract” refers to a bacterial cell lysate or a fraction thereof wherein the cellular extract is able to synthesis a protein from a nucleic acid template. In other words, the bacterial extract contains an energy source, such as ATP, GTP and the like. A bacterial extract can be a portion of a lysate from which other cellular components of the lysate have been separated by centrifugation, filtration, selective precipitation, selective immunoprecipitation, chromatography, or other methods. It also includes lysates or fractions thereof that contain exogenous material such as preservatives, stabilizers and reagents that enhance cell-free protein synthesis (CFPS). The term “bacterial extract” can refer to a preparation of an in vitro reaction mixture able to transcribe DNA into mRNA and/or translate mRNA into polypeptides. The mixture may include ribosomes, an energy source such as ATP, GTP, glucose, glutamate, or pyruvate, amino acids, and tRNAs. The mixture may be derived directly from lysed bacteria, from purified components or combinations of both.

The term “the extract is able to synthesize a target protein from a template nucleic acid encoding the target protein in cell-free protein synthesis” refers a lysed bacterial extract containing the necessary bacterial components needed to synthesize a protein of interest in a cell-free protein synthesis reaction.

“Cell-free protein synthesis” or “CFPS” refers to the in vitro synthesis of nucleic acids, polypeptides, small molecules and/or viral particles in a reaction mix comprising biological extracts and/or defined reagents. The reaction mix will comprise a template for production of the macromolecule, e.g., DNA, mRNA, etc.; monomers for the macromolecule to be synthesized, e.g., amino acids, nucleotides, etc.; and co-factors, enzymes and other reagents that are necessary for the synthesis, e.g., ribosomes, uncharged tRNAs, tRNAs charged with natural and/or unnatural amino acids, polymerases, transcriptional factors, tRNA synthetases, etc.

The term “spray dried bacterial extract” refers to a bacterial extract that has been spray dried as described herein. A “spray dried bacterial extract” is different from a “freeze-dried bacterial extract,” the latter of which refers to a bacterial extract that has been subjected to freeze drying, lyophilization, in situ vaporization, microwave radiation sublimation, and the like.

The term “stable, spray dried bacterial extract” refers to a spray dried bacterial extract that essentially retains its physical and chemical stability and integrity upon storage, for example, at −20° C., 2° C. to 8° C., or at room/ambient temperature (e.g., about 20° C.) for 6 months or more. For example, a stable, spray dried bacterial extract refers to an extract that retains at least 75% of its initial capacity to synthesize a protein of interest when stored at −20° C., 2° C. to 8° C., or at room/ambient temperature (e.g., about 20° C.) for 6 months or more.

The term “control bacterial extract” refers to a bacterial extract that is free of formulation additives, such as those described herein, or an extract that contains an additive described herein, but is tested at time T=0. Thus, a control bacterial extract can be an unformulated bacterial extract. The control extract can be an unformulated, spray dried bacterial extract. The control bacterial extract can be an unformulated, frozen bacterial extract. The control extract can be spray dried and/or stored at various temperatures, such as −80° C., −20° C., 4° C., 20° C., and 37° C. Alternatively, the control bacterial extract has not been spray-dried. In some instances, the control bacterial extract is a fresh bacterial extract. In some instances, the control bacterial extract is a liquid bacterial extract. The control extract can be a spray-dried extract with or without an additive described herein that is reconstituted prior to testing. The control extract can be a formulated spray-dried extract comprising an additive described herein that is tested at T=0.

The term “lysed bacterial components” refers to cellular components of a lysed bacterium. For example, the term can include bacterial components needed to synthesize a protein of interest from a template nucleic acid encoding the protein in a cell-free reaction, such as ribosomes, amino acids, polymerases, and tRNAs, and the components of an active oxidative phosphorylation system. Additional components can be added to the lysed bacterial components, such as exogenous ATP, GTP, glucose, glutamate, or pyruvate to provide an energy source.

The term “carbohydrate” refers to a macromolecule consisting of carbon, hydrogen, and oxygen atoms and having an empirical formula Cₘ(H2O)ₘ, wherein m and n may be different numbers. Carbohydrates include monosaccharides, disaccharides, oligosaccharides, and polysaccharides.

The term “rehydrating” or “reconstituting”, in the context of a spray dried bacterial extract, refers to suspending a spray dried bacterial extract in a diluent such as water or a buffer to disperse the components of the bacterial extract.

The term “water content” refers to the quantity of water contained in a material and can be expressed as a relative amount in percentage weight or volume.

The term “residual water” or “residual moisture” refers to the quantity of water contained in a material after the material has been processed, such as spray dried, and includes a range from about 1% to 15% residual water/residual moisture.

The term “protein synthesis activity” refers to the protein yield (e.g., the amount of protein) from a protein synthesis reaction to produce a target protein relative to a control protein synthesis reaction.

The term “lysate” is any cell derived preparation comprising the components required for protein synthesis machinery, wherein such cellular components are capable of expressing a nucleic acid encoding a desired protein where a majority of the biological components are present in concentrations resulting from the lysis of the cells rather than having been reconstituted. A lysate may be further altered such that the lysate is supplemented with additional cellular components, e.g., amino acids, nucleic acids, enzymes, etc. The lysate may also be altered such that additional cellular components are removed or degraded following lysis.

The terms “polypeptide,” “peptide” or “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds. The terms also encompass polymers comprising L-amino acids, polymers comprising D-amino acids, or polymers comprising both L- and D-amino acids.

“Non-natural” or “non-native” amino acid refers to amino acids that are not one of the twenty naturally occurring amino acids that are the building blocks for all proteins that are nonetheless capable of being biologically engineered such that they are incorporated into proteins. Non-native amino acids may include D-peptide enantiomers or any post-translational modifications of one of the twenty naturally occurring amino acids. A wide variety of non-native amino acids can be used in the methods of the disclosure. The non-native amino acid can be chosen based on desired characteristics of the non-native amino acid, e.g., function of the non-native amino acid, such as modifying protein biological properties such as toxicity, biodistribution, or half-life, structural properties, spectroscopic properties, chemical and/or photochemical properties, catalytic properties, ability to react with other molecules (either covalently or noncovalently), or the like. Non-native amino acids that can be used in the methods of the disclosure may include, but are not limited to, an non-native analogue of a tyrosine amino acid; an non-native analog of a glutamine amino acid; an non-native analog of a phenylalanine amino acid; an non-native analog of a serine amino acid; an non-native analog of a threonine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, phospho, phosphono, phosphine, heterocyclic, enone, imine, aldehyde, hydroxylamine, keto, or amino substituted amino acid, or any combination thereof; an amino acid with a photoactivatable cross-linker; a spin-labeled amino acid; a fluorescent amino acid; an amino acid with a novel functional group; an amino acid that covalently or noncovalently interacts with another molecule; a metal binding amino acid; a metal-containing amino acid; a radioactive amino acid; a photocaged and/or photoisomerizable amino acid; a biotin or biotin-analog containing amino acid; a glycosylated or carbohydrate modified amino acid; a keto containing amino acid; amino acids comprising polyethylene glycol or polyether; a heavy atom substituted amino acid; a chemically cleavable or photocleavable amino acid; an amino acid with an elongated side chain; an amino acid containing a toxic group; a sugar substituted amino acid, e.g., a sugar substituted serine or the like; a carbon-linked sugar-containing amino acid, e.g., a sugar substituted serine or the like; a carbon-linked sugar-containing amino acid; a redox-active amino acid; an alpha-hydroxy containing acid; an amino thio acid containing amino acid; an alpha,alpha-disubstituted amino acid; a beta-amino acid; a cyclic amino acid other than praline, etc.

The term “active oxidative phosphorylation system” in the context of a bacterial extract, refers to a bacterial extract that exhibits active oxidative phosphorylation during protein synthesis. For example, the bacterial extract can generate ATP using ATP synthase enzymes and reduction of oxygen. It will be understood that other translation systems known in the art can also use an active oxidative phosphorylation during protein synthesis. The activation of oxidative phosphorylation can be demonstrated by inhibition of the pathway using specific inhibitors, such as electron transport chain inhibitors.

As used in this application, an “increase” or a “decrease” refers to a detectable positive

or negative change in quantity from a comparison control, e.g., an established standard control (such as an extract that does not contain an additive or stabilizer). An increase is a positive change that is typically at least 10%, or at least 20%, or 50%, or 100%, and can be as high as at least 2-fold or at least 5-fold or even 10-fold of the control value. For example, the term “increased stability,” when used in relation to a bacterial extract described herein, refers to an extract having greater or more stability (i.e., greater or more protein synthesis activity) when stored at a given temperature for a given period of time relative to a control extract. Similarly, a decrease is a negative change that is typically at least 10%, or at least 20%, 30%, or 50%, or even as high as at least 80% or 90% of the control value. For example, the term “decreased stability,” when used in relation to a bacterial extract described herein, refers to an extract having less stability (i.e., less protein synthesis activity) when stored at a given temperature for a given period of time, and is usually associated with an extract that does not contain an additive or stabilizer of the disclosure. Other terms indicating quantitative changes or differences from a comparative basis, such as “more,” “less,” “higher,” and “lower,” as well as terms indicating an action to cause such changes or differences, such as “increase,” “promote,” “enhance,” “decrease,” “inhibit,” and “suppress,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

The term gram/liter (g/L) refers to a unit of measurement of mass concentration that shows how many grams of a substance are present in one liter of a liquid mixture.

The term gram/kilogram refers to a unit of a mass fraction expressed as a number of grams of a substance per kilogram of mixture.

### II. DETAILED DESCRIPTION OF EMBODIMENTS

Standard methods in molecular biology are described in Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3rd ed., Cold Spring Harbor Laboratory Press, Cold Spring Harbor, N.Y.; Wu (1993) Recombinant DNA, Vol. 217, Academic Press, San Diego, CA.). Standard methods also appear in Bindereif, Schőn, & Westhof (2005) Handbook of RNA Biochemistry, Wiley-VCH, Weinheim, Germany, which describes detailed methods for RNA manipulation and analysis, and Walker, J. M., (2009) The Protein Protocols Handbook, 3rd ed., Humana Press, New York, N.Y., which describes detailed methods for protein manipulation and analysis.

### A. Culturing Bacteria

Bacterial culturing is well known to those skilled in the art. A bacterial lysate derived from any strain of bacteria can be used in the methods of the disclosure. Bacteria suitable for use in cell-free synthesis systems include gram-negative bacteria and gram-positive bacteria, for example, Enterobacteriaceae such as Escherichia, e.g., E. coli, Enterobacter, Envinia, Klebsiella, Proteus, Salmonella, e.g., Salmonella typhimurium, Serratia, e.g., Serratia marcescans, and Shigella, as well as Bacilli such as B. subtilis and B. licheniformis, and Pseudomonas such as P. aeruginsa, and Steptomyces. In preferred embodiments, the bacteria used in the formulations and methods provided herein are from an Escherichia species, such as Escherichia coli or a derivative thereof.

The bacterial strain used to make the cell extract may have reduced nuclease and/or phosphatase activity which increases cell-free synthesis efficiency. For example, the bacterial strain used to make the cell-free extract can have mutations in the genes encoding the nucleases RNase E and RNase A. The strain may also have mutations to stabilize components of the cell synthesis reaction such as deletions in genes such as tnaA, speA, sdaA or gshA, which prevent degradation of the amino acids tryptophan, arginine, serine and cysteine, respectively, in a cell-free synthesis reaction. Additionally, the strain may have mutations to stabilize the protein products of cell-free synthesis such as knockouts in the proteases ompT or lonP.

The bacterial culture can be obtained as follows. The bacteria of choice are grown up overnight in any of a number of growth media and under growth conditions that are well known in the art and easily optimized by a practitioner for growth of the particular bacteria. In general, isolated strains of bacteria are grown in media until they reach balanced exponential growth phase or stationary phase. This can be between 10⁶ to 10⁹ cells per ml. In some embodiments, the culture is harvested when the pH of the culture rises above a set point indicating the depletion of glucose in the media. The bacterial culture can be grown to an OD₅₉₅₋₆₀₀ of 10 to 60, depending on the bacterial strain used. In some embodiments, the bacteria is cultured at a growth rate of about 0.06 to about 0.6 to about 0.8 doublings per hour.

The bacterial cells can be grown in medium containing glucose and phosphate, where the glucose is present at a concentration of at least about 0.25% (weight/volume), more usually at least about 1%; and usually not more than about 4%, more usually not more than about 2%. An example of such media is 2YTPG medium, however one of skill in the art will appreciate that many culture media can be adapted for this purpose, as there are many published media suitable for the growth of bacteria such as E. coli, using both defined and undefined sources of nutrients. Optimal media and growth conditions are known for specific species. For example, E. coli are commonly grown in YT broth (yeast extract and tryptone) or variants thereof. The media can be defined (synthetic) or complex (undefined).

Bacterial cells can be transfected or transformed with expression or cloning vectors and cultured in conventional nutrient media modified as appropriate for inducing promoters, selecting transformations and preparing bacterial extracts, as described herein.

In some instances, the bacteria are cultured in aerobic conditions to induce protein expression, and then the culture is switched to anaerobic conditions, for example by bubbling nitrogen, argon, etc. through the culture medium.

When large amounts of bacteria are needed, continuous culturing means are employed instead of batch systems which are closed. These continuous systems involve the continued introduction of nutrients and removal of waste. Optimally, this permits the cells to be grown at a constant biomass concentration for extended periods. Two well-known systems are chemostats and turbidostats. In the chemostat system sterile media is fed in at a constant rate while media containing bacteria is removed at the same rate. The turbidostat system uses a photocell to measure absorbance or turbidity and regulates the inflow of sterile media and outflow of bacteria according to preset signals.

Methods of culturing bacteria are described in, e.g., Zawada et al., Biotechnol. Bioeng., 108(7):1570-1578 (2011); Zawada, J. “Preparation and Testing of E.coli S30 In Vitro Transcription Translation Extracts”, Douthwaite, J. A. and Jackson, R. H. (eds.), Ribosome Display and Related Technologies: Methods and Protocols, Methods in Molecular Biology, vol. 805, pp. 31-41 (Humana Press, 2012); Jewett et al., Molecular Systems Biology: 4, 1-10 (2008); Shin J. and Norieaux V., J. Biol. Eng., 4:8 (2010).

In some instances, an engineered E. coli strain (e.g., engineered K-12 derived E. coli strain KGK10) is cultured to mid-log phase (OD₅₉₅ of about 45 OD or about 140 g/L of cell wet weight) using glucose and amino acid fed-batch fermentation at a maximal growth rate of about 0.7 h⁻¹. Glucose can be increased during culturing such that there is excess glucose during harvest. See, e.g., Zawada et al., Biotechnol. Bioeng., 108(7): 1570-1578 (2011).

### B. Preparing Bacterial Extracts

Once the bacterial culture is ready for harvest, it can be cooled to 2-8° C., usually on ice or through heat exchangers when the culture is of a large scale. The culture can be centrifuged to separate the spent media from the cell paste (cell slurry). Preferred centrifuges include disk stack centrifuges, tubular bowl centrifuges, and other centrifuges for large or small scale bacterial cultures. The cell paste is typically resuspended in S30 buffer, any equivalent buffer solution, or water. S30 buffer comprises 10 mM Tris acetate, 14 mM magnesium acetate and 60 mM potassium acetate. In some embodiments, a 1:1, 2:1, 3:1, 4:1, 5:1, 6:1, 7:1, 8:1 or more dilution (liquid: solid; ml of buffer: gram weight of cells) is made for washing. The cell paste can be washed again in S30 buffer or any equivalent buffer and centrifuged to remove any residual buffer. For small scale cultures, a second wash step is typically performed. At washing the cell paste (cell pellet) can be stored at −80° C. for use later or further processed by homogenization to lyse the cells.

A cell extract can be prepared from cultured bacteria, as described above. Cells that have been fermented overnight can be lysed by suspending the cell pellet in a suitable cell suspension buffer, and disrupting the suspended cells by sonication, breaking the suspended cells in a French press or with glass beads, continuous flow high pressure homogenization, or any other method known in the art useful for efficient cell lysis. The cell lysate is then centrifuged or filtered to remove large cell debris, including DNA, and cells that have not been lysed.

In some embodiments, the bacterial culture is pelleted by centrifugation at greater than 14,000×g for about 45 min at about 8-20° C. twice in a tubular bowl centrifuge in continuous or batch mode or a disc stack continuous centrifuge with a maximum bowl speed of about 12,000 rpm and a feed flow rate of about 3.0-3.3 L/min. The pelleted cells are resuspended and repelleted with S30 buffer. In some embodiments, the cells are stored at −80° C. for use later or processed by homogenization.

Prior to homogenization, the cell pellet can be resuspended in S30 buffer or an equivalent to produce a cell suspension. In some embodiments, a 1:1, 2:1, 3:1, 4:1, 5:1, 6:1, 7:1, 8:1 or more dilution (liquid: solid; ml of buffer: gram weight of cells) is created. Preferably, a 2:1 dilution is made such that 2 ml of S30 buffer is used per gram weight of cell pellet.

The cell suspension can be homogenized or disrupted in a standard high pressure homogenizer (e.g., an Avestin Emulsiflex C-55a Homogenizer) and/or microfluidizer (e.g., Microfluidics Microfluidizer) set at the appropriate pressure, such as 3,000 psi to produce a lysate. The homogenization step lyses the bacteria to release the necessary components required for protein synthesis, and in some aspects, formed inverted membrane vesicles provide energy for protein synthesis via respiration.

In some embodiments, the homogenizer pressure is at about 3,000-20,000 psi. In some embodiments, the homogenizer pressure is set at about 20,000 psi. In some embodiments, the speed (frequency setting) of the homogenizer is at about 20 Hz to about 60 Hz to produce flow rates of about 340 ml/min-1.0 L/min. Generally, flow rate is proportional to the frequency setting and can be varied independently from the homogenizing pressure. Preferably, the minimum speed setting for homogenizing steps is about 20 Hz with a flow rate of about 340 mL/min.

Bacterial lysates are also commercially available from manufacturers such Promega Corp., Madison, WI; Agilent Technologies, Santa Clara, CA; GE Healthcare Biosciences, Pittsburgh, PA; Life Technologies, Carlsbad, CA; and Roche Diagnostics, Basel, Switzerland.

Next, the lysate can be clarified by centrifugation such that from at least about 45% to about 85% or more, e.g., about 45%, 50%, 55%, 60%, 65%, 70%, 75%, 80%, 85% of the cell solids are separated from the cell-free extract which is collected. In some embodiments, at least about 70%, 75%, 80%, 85%, 90%, or 95% of the cellular solids are separated by centrifugation. In some embodiments, the centrifugation is by a continuous centrifuge, e.g., disk stack centrifuge, tubular bowl centrifuge or appropriate centrifuge. In some embodiments, 200 L fermentation yields greater than 1.1 L clarified extract/kg of cell wet weight with a total protein concentration of about 20-25 g/L.

The extract can be filtered through one or more sterilizing grade filter membranes, e.g., a 0.45 μm filter membrane and/or a 0.22 μm filter membrane. A 0.45 μm filter membrane can be used first, and then a 0.22 μm filter membrane afterwards.

In some embodiments, the filtered extract is activated or pre-incubated at 30° C. for about 2-5 hours, preferably for about 2.5 hours. After pre-incubation, particulates from the extract can be separated by centrifugation, e.g., spinning at least 14,000×g for about 35 minutes.

The lysed bacterial extract can be aliquoted and frozen in liquid nitrogen before storing at −80° C. Optionally, a cell-free synthesis reaction mix, as described herein, can be added to the cell-free extract prior to freezing.

Methods of preparing a lysed bacterial extract are described in, e.g., Zawada, J. “Preparation and Testing of E. coli S30 In Vitro Transcription Translation Extracts”, Douthwaite, J. A. and Jackson, R. H. (eds.), Ribosome Display and Related Technologies: Methods and Protocols, Methods in Molecular Biology, vol. 805, pp. 31-41 (Humana Press, 2012); Jewett et al., Molecular Systems Biology, 4, 1-10 (2008); Shin J. and Norieaux V., J. Biol. Eng., 4:8 (2010).

### C. Activation of Bacterial Extracts

The lysed bacterial extract prepared as above can be reconstituted in a buffer or other liquid to form a liquid bacterial extract. The liquid bacterial extract can be “activated” by heating the bacterial extract. In some embodiments, the liquid bacterial extract is heated to about 20° C. to 45° C. for about 30 minutes to about 10 hours. In some embodiments, the liquid bacterial extract is heated to about 40° C. for about 40 minutes. Activation improves protein expression in cell-free protein synthesis reactions. In some embodiments, the liquid bacterial extract is sterile filtered before activation (heat-treatment). Activation of bacterial extracts is described in Groff, D., et al. (Development of an E. coli strain for cell-free ADC manufacturing. Biotechnology and Bioengineering, 119, 162— 175. doi.org/10.1002/bit.27961).

Following activation, the liquid bacterial extract is formulated by adding the one or more additives as described herein below.

### D. Formulating Pre-Spray Dried Bacterial Extracts

The present disclosure is based, in part, on the unexpected result that specific additive(s), excipient(s), or stabilizer(s), when added to bacterial extracts prior to spray-drying, maintain the protein synthesis activity of the extract in cell-free protein synthesis reactions. Without being bound by theory, the additives may prevent denaturation of proteins during spray drying and/or long term storage. In some embodiments, the particular formulations of the stable, spray-dried bacterial extracts described herein can be stored for at least 8 months at −20° C., 2° C. to 8° C., or room temperature and have at least about 60% protein synthesis activity compared to a control extract with or without additives, excipients, or stabilizers. Experiments described in the Examples (see below) show that formulations with the additives described herein have long-term storage stability.

In some embodiments, the formulations comprise a single additive that is added to the bacterial extracts prior to spray-drying. Formulations comprising a single additive are referred to as “single component” spray-dried extracts.

In some embodiments, the formulations comprise a carbohydrate additive. In some embodiments, the carbohydrate additive is selected from trehalose, lactose, raffinose, maltodextrin, or cyclodextrin, or combinations thereof.

In some embodiments, the additive comprises trehalose (e.g., trehalose dihydrate (TDH); also known as D-(+)-Trehalose dihydrate). In some embodiments, the formulation comprises about 25 g/L to about 200 g/L trehalose, e.g., about 25, 30, 35, 40, 45, 50, 55, 60, 70, 75, 80, 85, 95, 100, 110, 120, 130, 140, 150, 160, 170, 180 190 or 200 g/L trehalose. In some embodiments, the formulation comprises about 50 g/L to about 100 g/L trehalose. In some embodiments, the formulation comprises about 25 g/L to about 75 g/L trehalose. In some embodiments, the formulation comprises about 75 g/L to about 125 g/L trehalose. In some embodiments, the formulation comprises about 50 g/L trehalose. In some embodiments, the formulation comprises about 75 g/L trehalose. In some embodiments, the formulation comprises about 100 g/L trehalose. In some embodiments, the formulation comprises about 125 g/L trehalose. In some embodiments, the formulation comprises about 25 g/kg to about 200 g/kg trehalose, e.g., about 25, 30, 35, 40, 45, 50, 55, 60, 70, 75, 80, 85, 90, 95, 100, 105, 110, 120, 130, 140, 150, 160, 170, 180, 190 or 200 g/kg trehalose. In some embodiments, the formulation comprises about 50 g/kg to about 110 g/kg trehalose. In some embodiments, the formulation comprises about 25 g/kg to about 75 g/kg trehalose. In some embodiments, the formulation comprises about 75 g/kg to about 125 g/kg trehalose. In some embodiments, the formulation comprises about 50 g/kg trehalose. In some embodiments, formulation comprises about 75 g/kg trehalose. In some embodiments, the formulation comprises about 100 g/kg trehalose. In some embodiments, the formulation comprises about 125 g/kg trehalose.

In some embodiments, the additive comprises lactose (e.g., lactose monohydrate (LMH)). In some embodiments, the formulation comprises about 25 to about 200 g/L lactose, e.g., about 25, 30, 35, 40, 45, 50, 55, 60, 70, 75, 80, 85, 90, 95, 100, 105, 110, 120, 130, 140, 150, 160, 170, 180, 190 or 200 g/L lactose. In some embodiments, the formulation comprises about 50 g/L to about 100 g/L lactose. In some embodiments, the formulation comprises about 100 g/L lactose. In some embodiments, the formulation comprises about 25 to about 200 g/kg lactose, e.g., about 25, 30, 35, 40, 45, 50, 55, 60, 70, 75, 80, 85, 90, 95, 100, 105, 110, 120, 130, 140, 150, 160, 170, 180, 190 or 200 g/kg lactose. In some embodiments, the formulation comprises about 50 g/L to about 100 g/kg lactose. In some embodiments, the formulation comprises about 100 g/kg lactose.

In some embodiments, the additive comprises raffinose. In some embodiments, the formulation comprises about 25 to 200 g/L raffinose, e.g., about 25, 30, 35, 40, 45, 50, 55, 60, 70, 75, 80, 85, 90, 95, 100, 105, 110, 120, 130, 140, 150, 160, 170, 180 190 or 200 g/L raffinose. In some embodiments, the formulation comprises about 25 to 200 g/kg raffinose, e.g., about 25, 30, 35, 40, 45, 50, 55, 60, 70, 75, 80, 85, 90, 95, 100, 105, 110, 120, 130, 140, 150, 160, 170, 180 190 or 200 g/kg raffinose.

In some embodiments, the additive comprises maltodextrin. In some embodiments, the formulation comprises about 100 g/L maltodextrin.

In some embodiments, the additive comprises cyclodextrin. Cyclodextrin helps to improve the aqueous solubility and stability of hydrophobic compounds. In some embodiments, the cyclodextrin is an alpha-, beta- or gamma-cyclodextrin, or combinations thereof. In some embodiments, the cyclodextrin is 2-Hydroxypropyl-β-Cyclodextrin (HP-β-CD; a cyclic oligosaccharide containing seven D-(+)-glucopyranose units). In some embodiments, the formulation comprises about 5 g/kg to about 50 g/kg cyclodextrin (e.g., about 5 g/kg to about 50 g/kg HP-β-CD).

In some embodiments, the formulations comprise an additive selected from a sugar alcohol. In some embodiments, the sugar alcohol is selected from mannitol or sorbitol, or a combination thereof. In some embodiments, the formulation comprises about 50 to about 100 g/L mannitol. In some embodiments, the formulation comprises about 15 g/L sorbitol.

In some embodiments, the formulations comprise an additive selected from an amino acid. In some embodiments, the amino acid is leucine. In some embodiments, the formulation comprises about 5 g/L to about 15 g/L leucine, e.g., about 5, 6, 7, 8, 9, 10, 11, 12, 13, 14, or 15 g/L leucine.

In some embodiments, the formulations comprise an additive selected from a polymer. In some embodiments, the polymer is selected from polyethylene glycol (PEG) (e.g., PEG 200, or a PEG having a molecular mass below 20,000 g/mol), polysorbate (e.g., polysorbate 20, 40, 60 or 80 (also known as Tween® 80), or polyvinylpyrrolidone (PVP or Kollidon 12 PF), or combinations thereof.

The additives can be mixed with a liquid lysed bacterial extract by combining a concentrated stock solution of one or more of the additives to achieve the preferred formulation of the spray-dried extract. For example, the liquid extract containing the additive(s) can have about 5 g/L-150 g/L, e.g., about 5 g/L, 10 g/L, 15 g/L, 20 g/L, 35 g/L, 40 g/L, 45 g/L, 50 g/L, 55 g/L, 60 g/L, 65 g/L, 70 g/L, 75 g/L, 80 g/L, 85 g/L, 90 g/L, 95 g/L, 100 g/L, 105 g/L, 110 g/L, 115 g/L, 120 g/L, 125 g/L, 130 g/L, 135 g/L, 140 g/L, 145 g/L, or 150 g/L of total dry weight of the additives per volume of lysed bacterial extract.

In some embodiments, the formulated bacterial extract does not comprise a composition, additive or stabilizer selected from sucrose, mannitol, sorbitol, dextran, or a combination thereof. In some embodiments, the formulated bacterial extract does not comprise sucrose.

### E. Combinations with Other Additives

The bacterial extract formulations described herein can include other compounds, such as additives, excipients, stabilizers, chemicals, molecules, or reagents that are added the lysed bacterial extract prior to spray drying. Thus, the bacterial extract can comprise a single additive described herein, or a single additive in combination with one or more other, different additives. Examples of other additives include carbohydrates, sugar alcohols, polymers, and/or amino acids, or combinations thereof. Representative other additives include raffinose, maltodextrin, sucrose, cyclodextrin (e.g., 2-Hydroxypropyl-β-Cyclodextrin), mannitol, sorbitol, PEG (e.g., polyethylene glycol 200), polysorbate (e.g., polysorbate 80 (Tween® 80)), polyvinylpyrrolidone (PVP or Kollidon 12 PF), leucine, amino acid mixtures, and/or combinations thereof.

Table 1 provides representative examples of single component and combination formulations tested for improved long-term stability as described in the Examples.

TABLE 1
Representative Single Component and Combination Formulations.

| No. | Formulations |
|---|---|
| 1 | Trehalose 25 g/L |
| 2 | Trehalose 50 g/L |
| 3 | Trehalose 75 g/kg |
| 4 | Trehalose 100 g/L |
| 5 | Trehalose 100 g/L + Leucine 8 g/L |
| 6 | Trehalose 100 g/L + PEG200 5 g/L |
| 7 | Trehalose 100 g/L + PEG200 15 g/L |
| 8 | Trehalose 100 g/L + PEG200 30 g/L |
| 9 | Trehalose 75 g/kg + Amino Acid Select Mix 10 g/kg |
| 10 | Trehalose 75 g/kg + 2-Hydroxypropyl-β-Cyclodextrin 25 g/kg |
| 11 | Lactose 100 g/L |
| 12 | Lactose 100 g/kg |
| 13 | Lactose 100 g/L + PEG200 15 g/L |
| 14 | Raffinose 90 g/L |
| 15 | Raffinose 90 g/L + PEG200 15 g/L |
| 16 | Lactose 100 g/kg + Amino Acid Select Mix 5 g/kg |
| 17 | Lactose 100 g/kg + Amino Acid Select Mix 10 g/kg |
| 18 | Lactose 100 g/kg + 2-Hydroxypropyl-β-Cyclodextrin 25 g/kg |
| 19 | Tween 80 0.1% |
| 20 | PVP (Polyvinylpyrrolidone; Kollidon 12 PF) 100 g/L |
| 21 | Leucine 8 g/L |
| 22 | Leucine 10 g/L |
| 23 | Maltodextrin 100 g/L |
| 24 | PMA Amino Acid Mix 13.03 g/L |
| 25 | Mannitol 50 g/L |
| 26 | Mannitol 80 g/L |
| 27 | Mannitol 100 g/L |
| 28 | Mannitol 100 g/L + Leucine 8 g/L |
| 29 | Mannitol 100 g/L + PEG200 15 g/L |
| 30 | Mannitol 100 g/L + PEG200 15 g/L + Leucine 8 g/L |
| 31 | Mannitol 100 g/L + PMA Amino Acid Mix 13.03 g/L |
| 32 | Mannitol 100 g/L + PEG200 15 g/L + PMA Amino Acid Mix |
| | 13.03 g/L |
| 33 | Mannitol 100 g/L + Sucrose 15 g/L + Sorbitol 15 g/L |

The difference between g/L and g/kg is density, such that 100 g/L TDH is equivalent to about 104 g/kg, and 75 g/kg TDH is equivalent to about 73 g/L.

In some embodiments, the bacterial extract formulations include one or more, a combination of all or a subset of the natural amino acids leucine, glycine, alanine, valine, isoleucine, proline, tryptophan, serine, threonine, methionine, asparagine, glutamine, cysteine, aspartic acid, glutamic acid, histidine, lysine, and arginine (referred to herein as “PMA amino acid mix”). A representative PMA amino acid mix is shown in Table 2.

TABLE 2
Representative PMA Amino Acid mix.

| Component | Concentration (g/L) |
|---|---|
| L-Valine | 0.624 |
| L-Tryptophan | 1.088 |
| L-Isoleucine | 0.699 |
| L-Leucine | 0.699 |
| L-Cysteine | 0.645 |
| L-Methionine | 0.795 |
| L-Alanine | 0.475 |
| L-Arginine | 0.928 |
| L-Asparagine monohydrate | 0.800 |
| L-Aspartic Acid | 0.709 |
| L-Glutamic Acid | 0.784 |
| Glycine | 0.400 |
| L-Glutamine | 0.779 |
| L-Histidine | 0.827 |
| L-Lysine HCl | 0.973 |
| L-Proline | 0.613 |
| L-Serine | 0.560 |
| L-Threonine | 0.635 |
| Total | 13.032 |

In some embodiments, the amino acids tyrosine and/or phenylalanine are added to the formulation separately from the PMA mix amino acids.

### F. High Glass Transition Components

In some embodiments, the bacterial extract formulation comprises one or more components having a high glass transition temperature (Tg). In some embodiments, the bacterial extract formulation comprises one or more components having a Tg of about 70° C. or more. For example, the bacterial extract formulation comprises one or more components having a Tg of about 80, 90 or 100° C. or more. In some embodiments, the bacterial extract formulation comprises trehalose. In some embodiments, the bacterial extract formulation comprises lactose. In some embodiments, the bacterial extract formulation comprises raffinose. The Tg of various components tested in the formulations of the disclosure are shown in the following Tables.

| Excipient | T_{g} (° C.) |
|---|---|
| Trehalose | 107, 115* |
| Lactose | 101 |
| Raffinose | 70 |
| Sucrose | 62, 70* |
| PVP | 170 |
| PEG200 | −48, −60 |
| Mannitol | 4.8 |
| Sorbitol | −9 |

| Amino Acid Code | Amino Acid | T_{g} (° C.) |
|---|---|---|
| Val | Valine | 658 |
| Asp | Aspartate | 399 |
| Ala | Alanine | 348 |
| Gly | Glycine | 326 |
| Trp | Tryptophan | 271 |
| His | Histidine | 215 |
| Glu | Glutamate | 214 |
| Pro | Proline | 150 |
| Cys-SH | Cysteine | 145 |
| Arg | Arginine | 137 |
| Leu | Leucine | 127 |
| Ile | Isoleucine | 127 |
| Met | Methionine | 89 |
| Thr | Threonine | 48 |
| Gln | Glutamine | 39 |
| Ser | Serine | 38 |
| Lys | Lysine | −15 |
| Asn | Asparagine | −41 |

*Note that for some components there are multiple sources that report slightly different values.

In some embodiments, the bacterial extract formulation comprises one or more, or a combination or sub-combination of high Tg nonpolar uncharged amino acids. Table 3 shows a representative combination of high Tg nonpolar uncharged amino acids used in some formulations (referred to herein as “Amino Acid Select Mix”).

TABLE 3
Representative High T_{g} Nonpolar Uncharged
Amino Acid (Amino Acid Select Mix).

| | Ratio of Amino | Mass | Mass |
|---|---|---|---|
| Component | Acid:Total | (g AA/kg extract) | (g AA/kg extract) |
| L-Valine | 0.14 | 0.68 | 1.36 |
| L-Tryptophan | 0.24 | 1.18 | 2.37 |
| L-Isoleucine | 0.15 | 0.76 | 1.52 |
| L-Leucine | 0.15 | 0.76 | 1.52 |
| L-Alanine | 0.10 | 0.52 | 1.03 |
| Glycine | 0.09 | 0.44 | 0.87 |
| L-Proline | 0.13 | 0.67 | 1.33 |
| Total | 1 | 5 | 10 |

In some embodiments, the bacterial extract comprises trehalose and one or more additives selected from a carbohydrate, a sugar alcohol, an amino acid or mixture of amino acids, a polymer, or a combination thereof. In some embodiments, the carbohydrate additive comprises cyclodextrin. In some embodiments, the concentration of cyclodextrin present in the bacterial extract is about 5 g/kg to about 50 g/kg (e.g., about 5 g/kg to about 50 g/kg HP-β-CD).

In some embodiments, the sugar alcohol is selected from mannitol or sorbitol, or a combination thereof. In some embodiments, the bacterial extract comprises about 50 to about 100 g/L mannitol. In some embodiments, the bacterial extract comprises about 15 g/L sorbitol.

In some embodiments, the bacterial extract comprises trehalose and an amino acid selected from (i) leucine; (ii) one or more of, a combination of all or a subset of the PMA amino acid mixture in Table 2; or (iii) one or more of, a combination of all or a subset of the amino acid select mix in Table 3. In some embodiments, the bacterial extract comprises trehalose and about 5 to 10 g/L leucine.

In some embodiments, the bacterial extract comprises trehalose and an additive selected from a polymer. In some embodiments, the polymer is selected from polyethylene glycol (PEG) (e.g., PEG 200, or a PEG having a molecular mass below 20,000 g/mol), polysorbate (e.g., polysorbate 20, 40, 60 or 80 (also known as Tween® 80), or polyvinylpyrrolidone (PVP or Kollidon 12 PF), or combinations thereof.

In any of the embodiments described herein, the trehalose can be trehalose dihydrate (TDH). In some embodiments, the concentration of trehalose or TDH present in the bacterial extract is about 25 to 200 g/L, e.g., about 25, 30, 35, 40, 45, 50, 55, 60, 70, 75, 80, 85, 90, 95, 100, 110, 120, 130, 140, 150, 160, 170, 180 190 or 200 g/L. In some embodiments, the bacterial extract comprises about 50 g/L to about 100 g/L trehalose or TDH. In some embodiments, the bacterial extract comprises about 100 g/L trehalose or TDH. In some embodiments, the bacterial extract comprises about 25 g/kg to about 200 g/kg trehalose, e.g., about 25, 30, 35, 40, 45, 50, 55, 60, 70, 75, 80, 85, 90, 95, 100, 105, 110, 120, 130, 140, 150, 160, 170, 180 190 or 200 g/kg trehalose. In some embodiments, the bacterial extract comprises about 50 g/kg to about 110 g/kg trehalose or TDH. In some embodiments, the bacterial extract comprises about 75 g/kg trehalose or TDH.

In some embodiments, the bacterial extract comprises lactose and one or more additives selected from a carbohydrate, a sugar alcohol, an amino acid or mixture of amino acids, a polymer, or a combination thereof. In some embodiments, the carbohydrate additive is selected from trehalose, raffinose, maltodextrin, sucrose, or cyclodextrin, or combinations thereof. In some embodiments, the concentration of trehalose present in the bacterial extract is about 25 g/kg to about 200 g/kg; the concentration of raffinose present in the bacterial extract is about 25 to 200 g/L; the concentration of maltodextrin present in the bacterial extract is about 100 g/L; and the concentration of cyclodextrin present in the bacterial extract is about 5 g/kg to about 50 g/kg (e.g., about 5 g/kg to about 50 g/kg HP-β-CD).

In some embodiments, the bacterial extract comprises lactose and a sugar alcohol selected from mannitol or sorbitol, or a combination thereof. In some embodiments, the bacterial extract comprises about 50 to about 100 g/L mannitol. In some embodiments, the bacterial extract comprises about 15 g/L sorbitol.

In some embodiments, the bacterial extract comprises lactose and an amino acid selected from (i) leucine; (ii) one or more, a combination of all or a subset of the PMA amino acid mixture in Table 2; or (iii) one or more, a combination of all or a subset of the amino acid select mix in Table 3. In some embodiments, the bacterial extract comprises lactose and about 5 to 10 g/L leucine.

In some embodiments, the bacterial extract comprises lactose and an additive selected from a polymer. In some embodiments, the polymer is selected from polyethylene glycol (PEG) (e.g., PEG 200, or a PEG having a molecular mass below 20,000 g/mol), polysorbate (e.g., polysorbate 20, 40, 60 or 80 (also known as Tween® 80), or polyvinylpyrrolidone (PVP or Kollidon 12 PF), or combinations thereof.

In any of the embodiments described herein, the lactose can be lactose monohydrate (LMH). In some embodiments, the bacterial extract comprises about 25 to about 200 g/L lactose or LMH, e.g., about 25, 30, 35, 40, 45, 50, 55, 60, 70, 75, 80, 85, 90, 95, 100, 105, 110, 120, 130, 140, 150, 160, 170, 180 190 or 200 g/L lactose or LMH. In some embodiments, the bacterial extract comprises about 100 g/L lactose or LMH. In some embodiments, the bacterial extract comprises about 25 to about 200 g/kg lactose, e.g., about 25, 30, 35, 40, 45, 50, 55, 60, 70, 75, 80, 85, 90, 95, 100, 105, 110, 120, 130, 140, 150, 160, 170, 180 190 or 200 g/kg lactose or LMH. In some embodiments, the bacterial extract comprises about 100 g/kg lactose or LMH.

In some embodiments, the bacterial extract comprises leucine and one or more additives selected from a carbohydrate, a sugar alcohol, an amino acid or mixture of amino acids, a polymer, or a combination thereof. In some embodiments, the carbohydrate additive comprises trehalose. In some embodiments, the concentration of trehalose present in the bacterial extract is about 25 g/kg to about 200 g/kg.

In some embodiments, the bacterial extract comprises leucine and a sugar alcohol selected from mannitol or sorbitol, or a combination thereof. In some embodiments, the bacterial extract comprises about 50 to about 100 g/L mannitol. In some embodiments, the bacterial extract comprises about 15 g/L sorbitol.

In some embodiments, the bacterial extract comprises leucine and an amino acid selected from (i) one or more of, a combination of all or a subset of the PMA amino acid mixture in Table 2; or (ii) one or more of, a combination of all or a subset of the amino acid select mix in Table 3.

In some embodiments, the bacterial extract comprises leucine and an additive selected from a polymer. In some embodiments, the polymer is selected from polyethylene glycol (PEG) (e.g., PEG 200, or a PEG having a molecular mass below 20,000 g/mol), polysorbate (e.g., polysorbate 20, 40, 60 or 80 (also known as Tween® 80), or polyvinylpyrrolidone (PVP or Kollidon 12 PF), or combinations thereof.

In some embodiments, the bacterial extract comprises raffinose and one or more additives selected from a sugar alcohol, an amino acid or mixture of amino acids, a polymer, or a combination thereof.

In some embodiments, the bacterial extract comprises raffinose and a sugar alcohol selected from mannitol or sorbitol, or a combination thereof. In some embodiments, the bacterial extract comprises about 50 to about 100 g/L mannitol. In some embodiments, the bacterial extract comprises about 15 g/L sorbitol.

In some embodiments, the bacterial extract comprises raffinose and an amino acid selected from (i) one or more of, a combination of all or a subset of the PMA amino acid mixture in Table 2; or (ii) one or more of, a combination of all or a subset of the amino acid select mix in Table 3. In some embodiments, the bacterial extract comprises raffinose and about 5 to 10 g/L leucine.

In some embodiments, the bacterial extract comprises raffinose and an additive selected from a polymer. In some embodiments, the polymer is selected from polyethylene glycol (PEG) (e.g., PEG 200, or a PEG having a molecular mass below 20,000 g/mol), polysorbate (e.g., polysorbate 20, 40, 60 or 80 (also known as Tween® 80), or polyvinylpyrrolidone (PVP or Kollidon 12 PF), or combinations thereof.

In some embodiments, the formulated bacterial extract prior to spray-drying does not comprise a composition, additive or stabilizer selected from sucrose, mannitol, sorbitol, dextran, or a combination thereof. In some embodiments, the formulated bacterial extract prior to spray-draying does not comprise sucrose.

### G. Methods for Producing a Stable, Spray-Dried Bacterial Extract

The bacterial extract comprising lysed bacterial components and one or more additive or stabilizer compositions described herein can then be spray dried. In some embodiments, the bacterial extract is a liquid (rehydrated or reconstituted) bacterial extract comprising lysed bacterial components for cell-free synthesis of a target protein from a template nucleic acid encoding the target protein.

In some embodiments, the method comprises atomizing the liquid bacterial extract to produce droplets; contacting the droplets with a gas to evaporate liquid from the droplets; separating the dried extract from the gas and smaller particles; and collecting the spray-dried extract.

In some embodiments, the disclosure provides methods for preparing a spray-dried bacterial extract, comprising providing a liquid bacterial extract comprising components for cell-free synthesis of a target protein from a template nucleic acid encoding the target protein; producing droplets of the liquid bacterial extract; contacting the droplets with a gas to evaporate liquid from the droplets; separating the dried extract from the gas and smaller particles; and collecting the spray-dried extract.

In some embodiments, the liquid bacterial extract comprising lysed bacterial components and one or more additive compositions is atomized by passing the liquid bacterial extract through an atomization device to produce droplets. In some embodiments, the atomization device is a tip, nozzle or rotary atomizer. In some embodiments, the tip or nozzle has an opening suitable for atomizing the liquid bacterial extract that is determined based on the type of drying equipment used and other factors by commercially appropriate means. In some embodiments, droplets of extract are produced using a two fluid nozzle system. The two fluid nozzle system may comprise a first nozzle that provides the liquid bacterial extract and a second nozzle that provides a pressurized gas that contacts the liquid bacterial extract as it exits the outlet of the first nozzle, thereby generating the droplets.

One of ordinary skill in the art will understand that the droplet size produced by the atomization device is dependent on several factors, including the atomization gas pressure, the desired liquid feed rate, the design of the nozzle or tip, and the drying equipment used. The drying gas flow rate, and the inlet and outlet temperatures of the drying gas may impact droplet size. These parameters can be adjusted based on the desired droplet size and residual moisture in the spray-dried extract. In some embodiments, the desired droplet size is from about 20 to 100 microns (Dv50). In some embodiments, the atomized gas pressure is from about 10 to 50 psig. In some embodiments, the outlet temperature of the drying gas is from about 60° C. to about 90° C. In some embodiments, the outlet temperature of the drying gas is from about 65° C. to about 80° C. For example, the outlet temperature of the drying gas may be about 65, 66, 67, 68, 69, 70, 71, 72, 73, 74, 75, 76, 77, 78, 79, or 80° C. In some embodiments, the outlet temperature of the drying gas is from about 65° C. to about 76° C.

It will be understood by a person of skill in the art that because the activity of the extract is temperature sensitive, the temperature used to dry the extract can be varied based on the size (volume) of the dryer used. For example, in larger, industrial-scale dryers the temperature may need to be decreased due to the longer residence time of the extract droplets in larger dryers compared to dryers with a smaller volume. It will be understood by a person of skill in the art that there are multiple ways to reduce the heat exposure in large dryers by special equipment design to shorten hold up time and/or to cool the powder particles faster once they are dried. Representative lab-scale dryers include a Buchi B-290 spray dryer. Representative pilot-scale dryers include a Mobile Minor PSD-1 spray dryer. Representative industrial-scale dryers include modified PSD-2 and PSD3 dryers, and an IFC-Welko spray dryer.

In some embodiments, the gas is dehumidified air. In some embodiments, the gas is nitrogen.

The spray-dried extract particles/droplets produced in the contacting step (ii) above can then be separated from the gas and any smaller particles using centrifugal force. In some embodiments, the spray-dried extract particles/droplets are separated from the gas and smaller particles using a cyclone. In some embodiments, the smaller particles are about 1 to 10 microns, and exit the cyclone in a separate stream from the desired spray-dried extract particles/droplets.

Following the separation step (iii) above, the spray-dried extract particles/droplets can then be collected. In some embodiments, spray-dried extract particles/droplets are collected in a container.

As noted above, prior to spray-drying, the liquid bacterial extract can be sterile filtered and activated by heating. In some embodiments, the liquid bacterial extract is activated by heating the extract to about 20 to 45° C. for about 30 minutes to about 10 hours.

In some embodiments, an additive or stabilizer composition comprising trehalose, lactose, leucine, or raffinose is added to the activated sterile filtered liquid bacterial extract prior to spray-drying (e.g., prior to the step of producing droplets of the liquid bacterial extract). In some embodiments, about 25 to 200 g/kg trehalose, about 25 to 200 g/kg (or about 25 to 200 g/L) lactose, about 5 to 10 g/L leucine, and/or about 25 to 200 g/L raffinose is added to the activated sterile filtered liquid bacterial extract prior to spray drying.

In some embodiments, one or more or a combination of amino acids are added to the activated sterile filtered liquid bacterial extract prior to spray drying. In some embodiments, the one or more amino acids comprise high glass transition temperate (Tg) nonpolar uncharged amino acids selected from the group consisting of L-Valine, L-Tryptophan, L-Isoleucine, L-Leucine, L-Alanine, Glycine, and L-Proline, and combinations thereof. In some embodiments, the one or more or combination of amino acids are selected from leucine, glycine, alanine, valine, isoleucine, proline, tryptophan, serine, threonine, methionine, asparagine, glutamine, cysteine, aspartic acid, glutamic acid, histidine, lysine, and/or arginine.

In some embodiments, one or more of maltodextrin, sucrose, mannitol, sorbitol, polyethylene glycol 200, polysorbate 80 (Tween® 80), polyvinylpyrrolidone (PVP or Kollidon 12 PF), and/or 2-Hydroxypropyl-β-Cyclodextrin, or combinations thereof, are added to the activated sterile filtered liquid bacterial extract prior to spray drying.

In some embodiments, greater than or equal to about 85% to 95% (w/w) (e.g., greater than or equal to about 85, 86, 87, 88, 89, 90, 91, 92, 93, 94, 95, 96, 97, 98, or 99% w/w) of the liquid is removed from the bacterial extract after spray-drying. In some embodiments, the spray-dried extract comprises less than or equal to about 15% (w/w) (e.g., less than or equal to about 15%, 14%, 13%, 12%, 11%, 10%, 9%, 8%, 7%, 6%, 5%, 4%, 3%, 2%, or 1% w/w) residual water.

### H. Residual Water Content

The amount of residual moisture or residual water can affect the protein synthesis activity and/or stability of the spray-dried extract formulations. In some embodiments, the spray-dried extract comprises less than about 15% residual moisture by weight, e.g., less than or equal to about 15%, 14%, 13%, 12%, 11%, 10%, 9%, 8%, 7%, 6%, 5%, 4%, 3%, 2% or 1% (w/w) residual moisture.

Methods of measuring water content in a dried extract include proton nuclear magnetic resonance (NMR) spectrometry and Karl Fisher coulometric titration.

NMR spectrometry is based on the fact that a hydrogen proton has a magnetic moment and an angular momentum. Hydrogen atoms produce a magnetic field when they are excited by an alternating field from a transmitter in the presence of the Earth's static magnetic field. The relaxation field is produced by the protons excited by the excitation field. The amplitude of the relaxation field measured after the excitation is turned off is directly related to the number of protons that have been excited and, thus, to the water content. Time-domain nuclear magnetic resonance (TD-NMR) spectrometry and “Spin Track” NMR spectrometry are variations of this technique that have been applied to biological cultures and protein solutions.

For the purposes of the disclosure provided herein, one method for measuring the percentage of water in a dried extract is Karl Fischer coulometric titration.

Karl Fischer titration utilizes the quantitative reaction of water with iodine and sulfur dioxide based on the Bunsen reaction in the presence of a primary alcohol such as methanol, ethanol or ethylene glycol monoethyl ether as the solvent, and an organic base such as pyridine as the buffering agent. Replacing the base with imidazole or primary amines may be utilized for a pyridine-free system. For protein or sugar solutions, a 2:1 methanol:formamide mixture may be used for solvent. Two variants in this method, the volumetric titration method and the coulometric titration method, utilize different iodine sources. In the volumetric titration method, the iodine required for reaction is previously dissolved and water content is determined by measuring the amount of iodine consumed as a result of reaction with water in a sample. Automatic volumetric titration systems are commercially available. In the coulometric titration method, iodine is first produced by electrolysis of a reagent containing iodide ion, then the water content is determined by measuring the quantity of electricity (Coulombs) [=electric current (Amperes)×time (seconds)] which is required for electrolysis in the production of iodine, based on the quantitative reaction of the generated iodine with water.

The Karl Fischer titration method can be performed using a drying oven (e.g., model D03080, Mettler Toledo, Columbus, OH) interfaced directly to a Karl Fisher coulometric titrator (e.g., model C20 from Mettler Toledo). Typically, the set point of the oven is set to 100° C. An aluminum insert is placed in the sample holder compartment of the oven and the extract to be measured is loaded into the insert through a port in the top of the oven. A nitrogen stream set at 200 mL/min is run through the oven to facilitate transfer of the water vapor from the oven to the titration vessel. The time between introduction of the sample to the oven and the start of the titration, or mix time, is set to 120 seconds to allow for complete transfer of the water in each sample to the titration vessel. The iodine for the titration is generated electrochemically in incremental amounts based on the drift observed by the instrument. The starting drift criterion is about less than about 25 μg/min. The drift criterion that should be achieved to end the measurement is less than about 3.0 μg/min, with a maximum titration time of about 3600 seconds. A voltametric sensor with a polarizing current of 5.0 μμA (e.g., model DM143-SC) is used for detection. Each sample can be run in triplicate in order to capture variability in the measurements.

### I. Spray-Dried Bacterial Extracts

The spray-dried bacterial extracts described herein can be used for cell-free protein synthesis reactions. In some embodiments, the spray-dried bacterial extract comprises dried, lysed bacterial components. In some embodiments, the spray-dried extract comprises components for synthesizing a target protein from a template nucleic acid encoding the target protein. In some embodiments, the spray-dried bacterial extract has an active oxidative phosphorylation system in cell-free protein synthesis. Additional components that are present in the spray-dried extract that can be used for cell-free protein synthesis are described below.

In some embodiments, the spray-dried bacterial extract comprises one or more of a stabilizer, wherein the stabilizer has a glass transition temperature (Tg) of at least about 70° C. In some embodiments, the stabilizer is selected from trehalose, lactose, leucine, and/or raffinose. In some embodiments, the liquid bacterial extract comprises about 25 to 200 g/kg trehalose, about 25 to 200 g/kg (or about 25 to 200 g/L) lactose, about 5 to 10 g/L of leucine, and/or about 25 to 200 g/L raffinose before spray-drying. In some embodiments, the trehalose is trehalose dihydrate (TDH) and the lactose is lactose monohydrate (LMH).

In some embodiments, greater than or equal to about 85% to 95% (w/w) (e.g., greater than or equal to about 85, 86, 87, 88, 89, 90, 91, 92, 93, 94, 95, 96, 97, 98, or 99% w/w) of the liquid is removed from the spray-dried bacterial extract. In some embodiments, the spray-dried bacterial extract comprises less than or equal to about 15% (w/w) (e.g., less than or equal to about 15%, 14%, 13%, 12%, 11%, 10%, 9%, 8%, 7%, 6%, 5%, 4%, 3%, 2%, or 1% w/w) residual water.

In some embodiments, the spray-dried bacterial extract further comprises one or more or a combination of high glass transition temperate (Tg) nonpolar uncharged amino acids selected from L-Valine, L-Tryptophan, L-Isoleucine, L-Leucine, L-Alanine, Glycine and/or L-Proline, and combinations thereof. In some embodiments, the spray-dried bacterial extract further comprises one or more, a combination of all, or a subset of amino acids selected from leucine, glycine, alanine, valine, isoleucine, proline, tryptophan, serine, threonine, methionine, asparagine, glutamine, cysteine, aspartic acid, glutamic acid, histidine, lysine, and arginine. In some embodiments, the spray-dried bacterial extract comprises about 5 to 15 g/L (or about 5 to 15 g/kg) of the amino acids.

In some embodiments, the spray-dried bacterial extract further comprises maltodextrin, sucrose, mannitol, sorbitol, polyethylene glycol 200, polysorbate 80 (Tween® 80), polyvinylpyrrolidone (PVP or Kollidon 12 PF), or 2-Hydroxypropyl-β-Cyclodextrin.

### J. Stability of the Spray-Dried Bacterial Extracts

The spray-dried extracts of the disclosure have increased stability when stored for long periods of time (e.g., 6 to 18 months or more) compared to extracts that do not contain the additives or stabilizers described herein. To determine stability, spray-dried extracts can be reconstituted by adding liquid to the spray-dried extract powder, and the reconstituted extract used in cell-free protein synthesis reactions. In some embodiments, the spray-dried extract is reconstituted (also referred to as rehydrated) by combining the spray-dried extract with a liquid such as a buffer or water. In some embodiments, a range of 5 to 10 g of liquid is added per one (1) g of dried extract powder (e.g., 5, 6, 7, 8, 9 or 10 g liquid per gram of spray-dried extract powder). In some embodiments, 5 g of water is added for every 1 g of spray dried extract. In some embodiments, 6 g of water is added for every 1 g of spray dried extract. In some embodiments, 7 g of water is added for every 1 g of spray dried extract. In some embodiments, 8 g of water is added for every 1 g of spray dried extract. In some embodiments, the reconstituted spray-dried extract is stored on ice or at about 0° C. to 8° C. prior to use in a CFPS reaction.

In some embodiments, the reconstituted or rehydrated spray-dried extract comprises about 20% to 60% (e.g., about 20%, 25%, 30%, 35%, 40%, 45%, 50%, 55% or 60%) by volume of the cell-free protein synthesis reaction.

The stability of the spray-dried extracts can be determined based on the yield (also expressed as titer) of a protein of interest produced by a cell-free protein synthesis reaction containing the spray-dried extract. In some embodiments, the yield of a protein of interest is determined as described below. In some embodiments, the yield of a protein of interest is determined by passing the cell-free protein synthesis reaction mixture through a Protein A resin or column. Protein A binds to proteins such as antibodies, and the bound protein of interest can then be eluted from the Protein A column. In some embodiments, a Protein A PhyTip® column (Biotage®) is used to purify and quantify the amount of protein of interest produced during a cell-free protein synthesis reaction.

In some embodiments, the spray-dried extract is stored at about −20° C. for at least 3, 6, 12, 18, 24, 30, 36 or greater than 36 months prior to being rehydrated. In some embodiments, the spray-dried extract is stored at about −20° C. for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or greater than 36 months prior to being rehydrated. In some embodiments, the rehydrated spray-dried extract that was stored at about −20° C. for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or greater than 36 months prior to rehydration is able to synthesize a target protein of interest with a yield of at least 80% relative to a control extract. In some embodiments, the rehydrated spray-dried extract that was stored at about −20° C. for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or greater than 36 months prior to rehydration is able to synthesize a target protein of interest with a yield of at least 85% relative to a control extract. In some embodiments, the rehydrated spray-dried extract that was stored at about −20° C. for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or greater than 36 months prior to rehydration is able to synthesize a target protein of interest with a yield of at least 90% relative to a control extract.

In some embodiments, the spray-dried extract is stored at about 2° C. to 8° C. for at least 3, 6, 12, 18, 24, 30, 36 or greater than 36 months prior to being rehydrated. In some embodiments, the spray-dried extract is stored at about 2° C. to 8° C. for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or greater than 36 months prior to being rehydrated. In some embodiments, the rehydrated spray-dried extract that was stored at about 2° C. to 8° C. for at least 3, 4, 5, 6, 7, 8, 9, 10, 11, 12, 13, 14, 15, 16, 17, 18 19, 20, 21, 22, 23, 24, 25, 26, 27, 28, 29, 30, 31, 32, 33, 34, 35, 36 or greater than 36 months prior to rehydration is able to synthesize a target protein of interest with a yield of at least 80% relative to a control extract. In some embodiments, the rehydrated spray-dried extract that was stored at about 2° C. to 8° C. for at least 3, 4, 5, 6, 7, 8, 9, 10, 11, 12, 13, 14, 15, 16, 17, 18 19, 20, 21, 22, 23, 24, 25, 26, 27, 28, 29, 30, 31, 32, 33, 34, 35, 36 or greater than 36 months prior to rehydration is able to synthesize a target protein of interest with a yield of at least 85% relative to a control extract. In some embodiments, the rehydrated spray-dried extract that was stored at about 2° C. to 8° C. for at least 3, 4, 5, 6, 7, 8, 9, 10, 11, 12, 13, 14, 15, 16, 17, 18 19, 20, 21, 22, 23, 24, 25, 26, 27, 28, 29, 30, 31, 32, 33, 34, 35, 36 or greater than 36 months prior to rehydration is able to synthesize a target protein of interest with a yield of at least 90% relative to a control extract.

In some embodiments, the spray-dried extract is stored at room temperature (RT) (e.g., about 20° C.) for at least 3, 6, 12 18, 24, 30, 36 or greater than 36 months prior to being rehydrated. In some embodiments, the spray-dried extract is stored at about 20° C. for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or greater than 36 months prior to being rehydrated. In some embodiments, the rehydrated spray-dried extract that was stored at about 20° C. for at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or greater than 36 months prior to rehydration is able to synthesize a target protein of interest with a yield of at least 60%, 65%, 70%, 75%, 80%, 85%, or 90% relative to a control extract.

In some embodiments, the control extract is a reconstituted unformulated extract, i.e., an extract that does not comprise an additive or stabilizer described herein. In some embodiments, the control extract is a frozen unformulated extract (stored at about −20° C. to −80° C.) that does not comprise an additive or stabilizer described herein. In some instances, the frozen unformulated control extract is a frozen liquid extract that was not previously dried. In other instances, the frozen unformulated control extract is a frozen liquid that is rehydrated from a spray-dried extract. In some embodiments, the control extract does not comprise an additive or stabilizer selected from trehalose, lactose, leucine, and raffinose.

In some embodiments, the control extract is a reconstituted formulated spray-dried extract that comprises an additive or stabilizer described herein. In some embodiments, the control extract is a reconstituted formulated spray-dried extract that comprises an additive or stabilizer selected from trehalose, lactose, leucine, and raffinose.

In some embodiments, the spray-dried bacterial extract and the spray-dried control extract comprise trehalose, and the spray-dried extract is stored at about −20° C., about 2° C. to 8° C., or about 20° C. (RT) for greater than or equal to 3 to 18 months (e.g., greater than or equal to 3, 4, 5, 6, 7, 8, 9, 10, 11, 12, 13, 14, 15, 16, 17, or 18 months). Following reconstitution, the spray-dried bacterial extract is able to synthesize the target protein of interest with a titer of at least 60%, 65%, 70%, 75%, 80%, 85%, 90%, or 95% relative to the control extract that was reconstituted at time zero. In some embodiments, the spray-dried bacterial and control extracts comprise about 100 g/L (about 105 g/kg) trehalose prior to rehydration, and the control extract is reconstituted at time T=0.

In some embodiments, the spray-dried extract and the control extract comprise about 100 g/L (about 105 g/kg) trehalose, the spray-dried extract is stored at about −20° C. for about 13.75 months, and following reconstitution is able to synthesize the target protein of interest with a titer of greater than about 90% compared to a control extract reconstituted at time T=0. In some embodiments, the spray-dried extract and the control extract comprise about 100 g/L (about 105 g/kg) trehalose, the spray-dried extract is stored at about 2° C. to 8° C. for about 13.75 months, and following reconstitution is able to synthesize the target protein of interest with a titer of about 80% compared to a control extract reconstituted at time T=0. In some embodiments, the spray-dried extract and the control extract comprise about 100 g/L (about 105 g/kg) trehalose, the spray-dried extract is stored at about 20° C. for about 13.75 months, and following reconstitution is able to synthesize the target protein of interest with a titer of greater than about 60 compared to a control extract reconstituted at time T=0.

In some embodiments, the protein synthesis activity of the rehydrated spray-dried extract is greater than or equal to the protein synthesis activity of rehydrated control spray-dried extract when the extracts are stored at 2° C. to 8° C. for greater than or equal to 8 months prior to rehydration. In some embodiments, the protein synthesis activity of the rehydrated spray-dried extract is greater than or equal to the protein synthesis activity of rehydrated control spray-dried extract when the extracts are stored at 2° C. to 8° C. for greater than or equal to 13 months prior to rehydration. In some embodiments, the protein synthesis activity of the rehydrated spray-dried extract is greater than or equal to the protein synthesis activity of rehydrated control spray-dried extract when the extracts are stored at about 20° C. (RT) for 8 months prior to rehydration. In some embodiments, the rehydrated spray-dried extract comprises trehalose. In some embodiments, the spray-dried extract comprises about 100 g/L (about 105 g/kg) trehalose prior to rehydration. In some embodiments, the control extract is an unformulated extract that does not comprise trehalose.

In some embodiments, the spray dried extract has a decrease in protein synthesis activity (as determined by rate of titer loss) of about 2% to about 6% per month when stored at about 2° C. to 8° C. prior to reconstitution. In some embodiments, the spray dried extract has a decrease in protein synthesis activity (as determined by rate of titer loss) of about 2% to about 6% per month when stored at 2 to 8° C. and a residual moisture content of less than or equal to about 7%, 6%, 5%, 4%, 3% or 2% (w/w) residual moisture.

In some embodiments, the spray dried extract comprises trehalose and residual moisture of about 2-6% and has a decrease in protein synthesis activity of about 2% to about 6% per month when stored at about 2° C. to 8° C. prior to reconstitution. In some embodiments, the spray dried extract comprises 100 g/L trehalose and residual moisture of about 2-3% and has a decrease in protein synthesis activity of about 2% to about 6% per month when stored at about 2° C. to 8° C. prior to reconstitution. In some embodiments, the spray dried extract comprises 75 g/L trehalose and residual moisture of about 2-3% and has a decrease in protein synthesis activity of about 4% to 5% per month when stored at 2° C. to 8° C. prior to reconstitution.

In some embodiments, the spray dried extract comprises lactose and residual moisture of about 3-4% and has a decrease in protein synthesis activity of about 3% to about 4% per month when stored at about 2° C. to 8° C. prior to reconstitution. In some embodiments, the spray dried extract comprises about 100 g/L or about 100 g/kg lactose and residual moisture of about 3-4%, and has a decrease in protein synthesis activity of about 3% to 4% per month when stored at 2° C. to 8° C. prior to reconstitution.

In some embodiments, the spray-dried extract comprises trehalose, is stored at 2° C. to 8° C. for about or at least 4 months prior to reconstitution and is able to synthesize the target protein of interest with a titer of at least 75% relative to an unformulated frozen control extract stored at −20° C. to −80° C. that does not comprise trehalose. In some embodiments, the spray-dried extract comprises about 70 to 100 g/L trehalose (e.g., 70, 75, 80, 85, 90, 95, or 100 g/L trehalose) prior to reconstitution. In some embodiments, the spray-dried extract comprises about 75 to 105 g/kg trehalose (e.g., 75, 80, 85, 90, 95, 100, or 105 g/kg trehalose) prior to reconstitution.

In some embodiments, the spray-dried extract comprises lactose, is stored at 2° C. to 8° C. for about or at least 4 months prior to reconstitution and is able to synthesize the target protein with a titer of at least 75% relative to an unformulated frozen control extract stored at −20° C. to −80° C. that does not comprise lactose. In some embodiments, the spray-dried extract comprises about 70 to 100 g/L lactose (e.g., 70, 75, 80, 85, 90, 95, or 100 g/L lactose) prior to reconstitution. In some embodiments, the spray-dried extract comprises about 75 to 105 g/kg lactose (e.g., 75, 80, 85, 90, 95, 100, or 105 g/kg lactose) prior to reconstitution.

### K. Using Spray Dried Bacterial Extracts in Cell-Free Protein Synthesis

Biologically active proteins of interest can be synthesized, properly folded and/or assembled using a cell-free protein synthesis system such as an Escherichia coli-based open cell-free (OCFS) system. In such a system, a cell extract from E. coli cells includes template DNA (such as plasmid or linear DNA fragments), amino acids (including native or non-native amino acids), nucleotides, T7 RNA polymerase, and an energy source. Optionally, disulfide isomerase chaperones is also added to aid in the formation of disulfide bonds. CFPS systems have been used to generate various proteins including growth factors (Zawada et al., Biotechnol Bioeng, 108:1570-1578 (2011)), full-length antibodies and antibody fragments (Yin et al., mAbs, 4(2):217-225 (2012)) and antibody-drug conjugates (Zimmerman et al., Bioconjug Chem, 25(2):351-61 (2014)).

The bacterial strain used to make the cell extract may have reduced nuclease and/or phosphatase activity, which increases cell-free synthesis efficiency. For example, the bacterial strain used to make the cell-free extract can have mutations in the genes encoding the nucleases RNase E and RNase A. The strain may also have mutations to stabilize components of the cell synthesis reaction such as deletions in genes such as tnaA, speA, sdaA or gshA, which prevent degradation of the amino acids tryptophan, arginine, serine and cysteine, respectively, in a cell-free synthesis reaction. Additionally, the strain may have mutations to stabilize the protein products of cell-free synthesis such as knockouts in the proteases ompT or lonP.

In a generic CFPS reaction, a gene encoding a protein of interest is expressed in a transcription buffer, resulting in mRNA that is translated into the protein of interest in a CFPS extract and a translation buffer. The transcription buffer, cell-free extract and translation buffer can be added separately, or two or more of these solutions can be combined before their addition or added contemporaneously.

To synthesize a protein of interest in vitro, the bacterial extract at some point comprises a mRNA molecule that encodes the protein of interest. In some systems, mRNA is added exogenously after being purified from natural sources or prepared synthetically in vitro from cloned DNA using RNA polymerases such as RNA polymerase II, SP6 RNA polymerase, T3 RNA polymerase, T7 RNA polymerase, RNA polymerase III and/or phage derived RNA polymerases.

In other systems, the mRNA is produced in vitro from a template DNA; both transcription and translation occur in this type of reaction. In some embodiments, the transcription and translation systems are coupled or comprise complementary transcription and translation systems, which carry out the synthesis of both RNA and protein in the same reaction. In such in vitro transcription and translation systems, the bacterial extracts contain all the components (exogenous or endogenous) necessary both for transcription (to produce mRNA) and for translation (to synthesize protein) in a single system.

A CFPS reaction mixture can contain the following components: a template nucleic acid, such as DNA, that comprises a gene of interest operably linked to at least one promoter and, optionally, one or more other regulatory sequences (e.g., a cloning or expression vector containing the gene of interest) or a PCR fragment; an RNA polymerase that recognizes the promoter(s) to which the gene of interest is operably linked (e.g. T7 RNA polymerase) and, optionally, one or more transcription factors directed to an optional regulatory sequence to which the template nucleic acid is operably linked; ribonucleotide triphosphates (rNTPs); optionally, other transcription factors and co-factors therefor; ribosomes; transfer RNA (tRNA); other or optional translation factors (e.g., translation initiation, elongation and termination factors) and co-factors therefore; one or more energy sources, (e.g., ATP, GTP); optionally, one or more energy regenerating components (e.g., PEP/pyruvate kinase, AP/acetate kinase or creatine phosphate/creatine kinase); optionally factors that enhance yield and/or efficiency (e.g., nucleases, nuclease inhibitors, protein stabilizers, chaperones) and co-factors therefore; and; optionally, solubilizing agents. The reaction mix can also include amino acids and other materials specifically required for protein synthesis, including salts (e.g., potassium, magnesium, ammonium, and manganese salts of acetic acid, glutamic acid, or sulfuric acids), polymeric compounds (e.g., polyethylene glycol, dextran, diethyl aminoethyl dextran, quaternary aminoethyl and aminoethyl dextran, etc.), cyclic AMP, inhibitors of protein or nucleic acid degrading enzymes, inhibitors or regulators of protein synthesis, oxidation/reduction adjuster (e.g., DTT, ascorbic acid, glutathione, and/or their oxides), non-denaturing surfactants (e.g., Triton X-100) , buffer components, spermine, spermidine, putrescine, etc. Components of such reactions are discussed in more detail in U.S. Pat. Nos. 7,338,789; 7,351,563; 8,715,958; and 8,778,631, the disclosures of each are incorporated by reference in their entirety for all purposes.

Depending on the specific enzymes present in the extract, for example, one or more of the many known nuclease, polymerase or phosphatase inhibitors can be selected and advantageously used to improve synthesis efficiency.

Protein and nucleic acid synthesis typically requires an energy source. Energy is required for initiation of transcription to produce mRNA (e.g., when a DNA template is used and for initiation of translation high energy phosphate for example in the form of GTP is used). Each subsequent step of one codon by the ribosome (three nucleotides; one amino acid) requires hydrolysis of an additional GTP to GDP. ATP is also typically required. For an amino acid to be polymerized during protein synthesis, it must first be activated. Significant quantities of energy from high energy phosphate bonds are thus required for protein and/or nucleic acid synthesis to proceed.

An energy source is a chemical substrate that can be enzymatically processed to provide energy to achieve desired chemical reactions. Energy sources that allow release of energy for synthesis by cleavage of high-energy phosphate bonds such as those found in nucleoside triphosphates, e.g., ATP, are commonly used. Any source convertible to high energy phosphate bonds is especially suitable. ATP, GTP, and other triphosphates can normally be considered as equivalent energy sources for supporting protein synthesis.

To provide energy for the synthesis reaction, the system can include added energy sources, such as glucose, pyruvate, phosphoenolpyruvate (PEP), carbamoyl phosphate, acetyl phosphate, creatine phosphate, phosphopyruvate, glyceraldehyde-3-phosphate, 3-Phosphoglycerate and glucose-6-phosphate, that can generate or regenerate high-energy triphosphate compounds such as ATP, GTP, other NTPs, etc.

When sufficient energy is not initially present in the synthesis system, an additional source of energy is preferably supplemented. Energy sources can also be added or supplemented during the in vitro synthesis reaction.

In some embodiments, the cell-free protein synthesis reaction is performed using the PANOx-SP system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, phosphoenol pyruvate (PEP), NAD, CoA, Na⁺ oxalate, putrescine, spermidine, and S30 extract.

In some embodiments, proteins containing a non-natural amino acid (nnAA) may be synthesized. In such embodiments, the reaction mix may comprise the non-natural amino acid, a tRNA orthogonal to the 20 naturally occurring amino acids, and a tRNA synthetase that can link the nnAA with the orthogonal tRNA. See, e.g., U.S. Pat. No. 8,715,958. Alternatively, the reaction mix may contain a nnAA conjugated to a tRNA for which the naturally occurring tRNA synthetase has been depleted. See, e.g., U.S. Pat. No. 8,778,631 and U.S. App. Publ. No. 2010/0184134. Various kinds of unnatural amino acids, including without limitation detectably labeled amino acids, can be added to cell-free protein synthesis reactions and efficiently incorporated into proteins for specific purposes. See, for example, Albayrak, C. and Swartz, J R., Biochem. Biophys Res. Commun., 431(2):291-5; Yang W C et al., Biotechnol. Prog., (2012), 28(2):413-20; Kuechenreuther et al., PLoS One, (2012), 7(9):e45850; and Swartz J R., AIChE Journal, 58(1):5-13.

In some instances, the cell-free synthesis reaction does not require the addition of commonly secondary energy sources, yet uses co-activation of oxidative phosphorylation and protein synthesis. In some instances, CFPS is performed in a reaction such as the Cytomim (cytoplasm mimic) system. The Cytomim system is defined as a reaction condition performed in the absence of polyethylene glycol with optimized magnesium concentration. This system does not accumulate phosphate, which is known to inhibit protein synthesis. Detailed descriptions of the Cytomim system are found in, for example, U.S. Pat. No. 7,338,789; Jewett et al., Mol Syst Biol, (2008), 4:220; Spirin, A. S. and Swartz, J. R. (2008) Cell-free Protein Synthesis; Methods and Protocols, New Jersey:John Wiley & Sons, the contents are hereby incorporated in their entirety for all purposes.

The presence of an active oxidative phosphorylation pathway can be tested using inhibitors that specifically inhibit the steps in the pathway, such as electron transport chain inhibitors. Examples of inhibitors of the oxidative phosphorylation pathway include toxins such as cyanide, carbon monoxide, azide, carbonyl cyanide m-chlorophenyl hydrazone (CCCP), and 2,4-dinitrophenol, antibiotics such as oligomycin, pesticides such as rotenone, and competitive inhibitors of succinate dehydrogenase such as malonate and oxaloacetate.

In some embodiments, the cell-free protein synthesis reaction is performed using the Cytomim system comprising NTPs, E. coli tRNA, amino acids, Mg²⁺ acetate, Mg²⁺ glutamate, K⁺ acetate, K⁺ glutamate, folinic acid, Tris pH 8.2, DTT, pyruvate kinase, T7 RNA polymerase, disulfide isomerase, sodium pyruvate, NAD, CoA, Na⁺ oxalate, putrescine, spermidine, and S30 extract. In some embodiments, the energy substrate for the Cytomim system is pyruvate, glutamic acid, and/or glucose. In some embodiments of the system, the nucleoside triphosphates (NTPs) are replaced with nucleoside monophosphates (NMPs).

The cell extract can be treated with iodoacetamide in order to inactivate enzymes that can reduce disulfide bonds and impair proper protein folding. As further described herein, the cell extract can also be treated with a prokaryotic disulfide bond isomerase, such as, not limited to, E. coli DsbC and PDI. The cell extract can be treated with DsbC, FkpA and peptidyl peolyl isomerase. Exogenous chaperone proteins can be expressed by the bacteria strain of the cell extract. Glutathione disulfide (GSSG) and glutathione (GSH) can also be added to the extract at a ratio that promotes proper protein folding and prevents the formation of aberrant protein disulfides.

In some embodiments, the CFS reaction includes inverted membrane vesicles to perform oxidative phosphorylation. These vesicles can be formed during the high pressure homogenization step of the preparation of cell extract process, as described herein, and remain in the extract used in the reaction mix.

The cell-free synthesis reaction conditions may be performed as batch, continuous flow, or semi-continuous flow, as known in the art. The reaction conditions are linearly scalable, for example, the 0.3 L scale in a 0.5 L stirred tank reactor, to the 4 L scale in a 10 L fermentor, and to the 100 L scale in a 200 L fermentor.

The protein synthesis reactions described herein can utilize a large scale reactor, small scale, or may be multiplexed to perform a plurality of simultaneous syntheses. Continuous reactions can use a feed mechanism to introduce a flow of reagents, and may isolate the end-product as part of the process. Batch systems are also of interest, where additional reagents may be introduced to prolong the period of time for active synthesis. A reactor can be run in any mode such as batch, extended batch, semi-batch, semi-continuous, fed-batch and continuous, and which will be selected in accordance with the application purpose.

### L. Methods for Comparing the Yield of Cell-Free Protein Synthesis

The activity (e.g., yield of a specific protein in a cell-free protein synthesis system) of the dried extract can be determined using assays such as performing cell-free protein synthesis to produce a model protein (test protein) which can be measured. Methods for cell-free protein synthesis are described in detail in, e.g., Kim, D. M. and Swartz, J. R. Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al., Methods Mol. Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005).

The amount of protein produced in a CFPS reaction can be measured using any method known to one of skill in the art. In some embodiments, the yield of a protein of interest is determined by dual flow chromatography (DFC) using a Protein A resin. The Protein A resin can be packed between two thin frit screens in the tip of a single-use pipette tip. In some embodiments, the yield of a protein of interest is determined by passing the products of a cell-free synthesis reaction over a Protein A column, such as a PhyTip® column (Biotage®). The yield of a protein of interest can be expressed as weight/volume (e.g., mg/L or g/L) of a CFPS reaction or as a percentage of the yield of a control extract.

In some embodiments, the yield of a protein of interest is determined by performing High-performance liquid chromatography (HPLC) of the protein products from a CFPS reaction along with protein standards.

In some embodiments, the yield of a protein of interest is determined by an assay which measures the activity of the particular protein being translated. An example of an assay for measuring protein activity is a luciferase assay system, or chloramphenicol acetyl transferase assay system for the production of the associated proteins. These assays measure the amount of functionally active protein produced from the translation reaction. Assays for measuring protein levels include, but are not limited to, coomassie-stained polyacrylamide gel, silver-stained polyacrylamide gel, ELISA, immunoblotting, Western blotting, size exclusion chromatography, affinity chromatography, and mass spectrometry. The activity of the particular protein being translated can be measured using any method known to one of skill in the art measures the activity (e.g., function) of the particular protein of interest. For example, the amount of particular kinase produced in a translation reaction can be measured by a kinase assay, wherein the activity of the particular kinase is determined by quantifying a kinase reaction.

Another method of measuring the amount of protein produced in coupled in vitro transcription and translation reactions is to perform the reactions using a known quantity of radiolabeled amino acid such as ³⁵S-methionine, ³H-leucine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

Methods of measuring the capacity of an expression system to express a protein includes the ¹⁴C Leu incorporation assay. In some embodiments, a method for measuring the protein synthesis activity of a spray-dried extract is the ¹⁴C Leu incorporation assay.

In some embodiments, the yield of soluble protein is calculated from the amount of ¹⁴C Leu incorporated into soluble proteins produced in a cell-free protein synthesis reaction. The extract can be treated with 50 μM iodoacetimide (IAM) for about 30 minutes at room temperature. IAM is added to allow for the formation of disulfide bonds within the protein of interest. Other thiol capping reagents such as iodoacetic acid (IAA) and N-ethyl maleimide (NEM) can be substitute for IAM.

Typically, the extract is then added to a microcentrifuge tube containing a protein synthesis reaction mixture with ¹⁴C Leu in order to initiate the reaction. About 60 μl of the reaction mix is transferred to a 24-well plate and spread evenly about the well. The mix is incubated at 30° C. for 5 hours. At the end of 5 hours the mix is transferred to a new microfuge tube. Two 10 μl aliquots are transferred to two slips of chromatography paper labeled “A” and “B”. “A” represents total counts and “B” represents total protein counts. The remaining mix in the tube is centrifuged for about 15 minutes in a microfuge at about 13,000 rpm. Two 10 μl aliquots of the supernatant are transferred to two slips of chromatography paper labeled “C” and “D”. “C” and “D” represent soluble protein. All slips of paper are dried at about 2 inches from a heat lamp for about 15 minutes. “A” is transferred to a microcentrifuge tube. “B”, “C” and “D” are washed 3× with 5% TCA on ice for about 15 minutes, and then washed with 100% ethanol. They are then dried under a heat lamp for about 15 minutes. “B”, “C” and “D” slip are transferred to individual microcentrifuge tubes. Scintillation cocktail (Optiphase Supermix, PerkinElmer, Waltham, MA) is added to each microcentrifuge and the slips are counted in a scintillation counter for 5 minutes.

The yield of total protein can be determined by the following equation:

(Counts slip B/counts slip A) (leucine concentration in cell free/# of leucines in protein of interest) (MW of protein of interest)

The yield of soluble protein of “C” and “D” can be determined by the following equation:

(Counts slip C or D/counts slip A)(conc of leucine in CF/# leu residues in protein of interest)(MW of protein of interest)

The average yield of soluble protein can be determined by averaging the yield of “C” and “D”.

Alternately, the yield of protein can be determined by running the protein labeled with ¹⁴C Leu on a polyacrylamide gel using conventional techniques. The gel can be denaturing or non-denaturing, according to the polypeptide to be detected. Where a protein containing multiple subunits is to be detected, a non-denaturing gel is preferred.

Alternatively, the yield of protein can be determined through specific binding assays such as enzyme linked immunosorbant assay (ELISA) or surface binding resonance (e.g., Biacore).

Alternatively, the yield of protein can be determined through whole or partial purification, such as using chromatography, coupled with protein quantitation, such as UV absorbance or BCA analysis.

Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

### EXAMPLES

### Example 1. Preparation of Bacterial Cell-Free Extracts

This example provides a representative method for preparing bacterial cell-free extracts. Exemplary methods for the preparation of bacterial cell-free synthesis extracts are described in Zawada, J. F., et al. (Microscale to manufacturing scale-up of cell-free cytokine production—a new approach for shortening protein production development timelines. Biotechnol Bioeng. 2011 Jul;108(7):1570-8. doi: 10.1002/bit.23103) and Groff, D., et al. (Development of an E. coli strain for cell-free ADC manufacturing. Biotechnology and Bioengineering, 119, 162— 175. https://doi.org/10.1002/bit.27961).

For preparation of bacterial extracts on a small scale, 10 μl of a thawed glycerol stock of E. coli was used to inoculate 50 mL of 2YT medium in a 250 mL baffled flask. The culture was incubated overnight at 37° C. with vigorous shaking. The 50 mL culture was then transferred into 1 L of 2YTPG medium in a 2.5 L flask with a filter lining in the cap. The culture was incubated at 30° C. with vigorous shaking and monitored for growth rate. During the exponential phase and before the growth rate dropped during the transition to stationary phase, the cells were harvested and chilled. Once the culture was chilled, the cells were collected by centrifugation at 8,000×g for 20-30 minutes. Approximately 8 g of wet cells were collected from 1 L at an OD of 3. The cell pellet was resuspended in at least 5 mL of S30 buffer for each gram of wet cell weight. The cell suspension was centrifuged at 8,000×g for 20-30 minutes. The supernatant was discarded and the washed cell pellet was frozen at −80 ° C.

For preparation of bacterial extracts on a large scale, the culture was harvested after 16-20 hours of fermentation. The fermenter (200 L fermenter) was then pressurized to 20 psi and the culture, approximately 200 L, was transferred, via pressure, to a chilled 200 L jacketed holding tank through two heat exchangers connected in parallel. The culture temperature in the holding tank, approximately 200 L, was cooled to 2-8° C. by re-circulating glycol through the jacket. Following the completion of the transfer, the culture was ready for the first centrifugation step via the disk stack centrifugation. During the first centrifugation step, the cells were separated from the spent medium (supernatant). The discharged paste (cell slurry) was collected into a container, weighed and then dispensed into a chilled 200 L holding tank containing 100 L of S30 buffer and mixed to re- suspend. The re-suspended cells were then centrifuged to pellet the cells. The cell pellet was stored at −80 ° C.

One liter of 2YTPG medium contains 16 g/L tryptone, 10 g/L yeast extract, 5 g/L sodium chloride, 22 mM sodium phosphate monobasic, 40 mM sodium phosphate dibasic, 100 mM glucose, and optionally 100 μl antifoam 204. 2YT medium contains 16 g/L tryptone, 10 g/L yeast extract, and 5 g/L sodium chloride. S30 buffer comprises 10 mM Tris acetate, 14 mM magnesium acetate and 60 mM potassium acetate.

Frozen S30 cell paste was broken into small pieces and thawed in 1 mL of room temperature S30 buffer per gram of cell paste. Once thawed, the cell suspension was kept on ice. The cell homogenizer was rinsed in S30 buffer prior to processing the cell extract. The cells were lysed by a single pass through the high pressure cell homogenizer at 17,500 psi. The lysate was then cooled quickly through a cooling coil or heat exchanger. The lysate was kept on ice until all the cell paste was lysed as described herein. The lysate was centrifuged at 30,000×g for 30 minutes at 4° C. The supernatant was collected into a clean tube and the centrifugation step was repeated once again in order to collect all supernatant in the lysate.

The collected supernatant has 0.2 mL pre-incubation mix added for each 1 mL supernatant. Then the mixture is incubated at about 37° C. for about 80 minutes. The pre-incubation mix contained 370 mM Tris acetate pH 8.2, 11.1 mM magnesium acetate, 16.5 mM ATP, 50 μM each of the 20 amino acids or non-native amino acids, 105 mM phosphoenol pyruvate (PEP), and 8.4 U/mL pyruvate kinase.

### Example 2. Formulating Cell-Free Extracts with Additives

This example provides a representative method for formulating bacterial cell-free extracts with additives that are useful for stabilizing a spray-dried extract.

Liquid bacterial extract was formulated either by directly mixing in powdered excipients, or by addition of concentrated stock solutions of additives to achieve various additive levels and combinations in the formulated extract prior to spray drying. The concentrated stock solutions can be sterile filtered prior to being adding to the liquid extract for bioburden control. Some excipients exhibited facile aqueous dissolution at ambient and chilled conditions at concentrations up to several hundred grams per liter, while others had limited solubility by comparison. Some components exhibited solubility in extract up to at least 100 g/L or 100 g/kg and were tested at these levels, while lower extract solubility limits determined maximum concentrations that could be evaluated for some other excipients. The additives used included trehalose dihydrate (TDH), lactose monohydrate (LMH), raffinose, maltodextrin, sucrose, mannitol, sorbitol, polyethylene glycol 200, polysorbate 80 (Tween® 80), polyvinylpyrrolidone (PVP or Kollidon 12 PF), and 2-Hydroxypropyl-β-Cyclodextrin. Over 33 formulations were created and dried to evaluate extract formulations. Representative single component and combination formulations are shown in Table 1.

Additionally, some mixtures included a combination of all or a subset of the natural amino acids leucine, glycine, alanine, valine, isoleucine, proline, tryptophan, serine, threonine, methionine, asparagine, glutamine, cysteine, aspartic acid, glutamic acid, histidine, lysine, and arginine, referred herein as “PMA amino acid mix.” The PMA amino acid mix optionally does not include tyrosine or phenylalanine. In some embodiments, tyrosine and/or phenylalanine are added separately from the PMA mix in the XCF reaction mixture. A representative PMA amino acid mix is shown in Table 2.

### Example 3. Spray Drying Process

This example describes a representative spray-drying process.

Spray drying is a scalable continuous process that converts a liquid feedstock into a fine powder with a reduction in material volume and bulk density. Spray drying of extract, such as XtractCF®, was achieved via (1) atomization of the liquid feedstock material into a fine mist by pumping it through an atomization device, e.g., a nozzle or a rotary atomizer, (2) intersection of that fine mist with a convective flow of hot dry gas in a chamber that evaporated the liquid to dry it (3) separation of dried product material from humid drying gas and smaller fine particulates using a cyclone or other similar technology designed for powder fractionation, and (4) dried material collection. Spray drying of XtractCF® from liquid to powdered form enables reduced material volume and enhanced storage capabilities necessary for commercial scale production.

The liquid feedstock material was formulated prior to spray drying, and comprised of extract solids, formulation solids, and water. The formulated feedstock was continuously pumped to the dryer at a liquid feed rate determined by the dryer process control scheme. The material was fed through tubing into a two-fluid nozzle. The nozzle combined the liquid with an atomization gas stream that had an atomization gas pressure and atomization gas flow rate which influenced the droplet size formed based on the pressure and relative flow of the two streams. Once atomized into a fine mist, the droplets were contacted with the drying gas that was at the inlet temperature. Evaporative cooling occurred, however, and the solids did not directly experience the high inlet drying gas temperature. The mist was exposed to the drying gas for the duration of the residence time in the drying chamber during which time the drying gas cooled to the outlet temperature. Initially the solids in the droplets experienced the wet bulb temperature, but as the water evaporated, there was a transition to where the solids experienced the dry bulb temperature. An interdependency between multiple of the aforementioned process parameters and outputs existed in the spray drying process, and the balance of the momentum, heat, and mass transfer in the process defined by these parameters determined the particle size or particle size distribution which influenced the yield. The water evaporation during drying was significant. The dried material contained a portion of residual moisture or water content, which showed negative correlation with observed shelf-life stability. The spray-dried extract typically has less than 7% residual moisture content and can have less than 5% residual moisture content.

For XtractCF® spray drying, the inlet and outlet temperatures had a significant impact on XpressCF® performance. The residual moisture or water content of the spray dried XtractCF® was observed to have a significant impact on shelf-life stability for storage at 2-8° C. and ambient temperature. Development of XtractCF® spray drying has included work on lab-scale (Buchi B-290), pilot-scale (Mobile Minor PSD-1), and industrial-scale (modified PSD-2, PSD3, and IFC Welko≈PSD-4). These drying scales span batch sizes from ˜0.1 L-1000 L and throughputs of ˜1 kg/hr-100 kg/hr. Larger dryers typically have longer material residence times, which can result in greater heat exposure to XtractCF® during the drying process. As a result, adjustments to lower the temperature setpoints, for the outlet temperature specifically, were made to maintain optimal XpressCF® activity in larger scale drying. The outlet temperature for larger dryers was adjusted to about 65 to 76° C.

Process control schemes can take on multiple configurations, whereby some parameters are controlled around a setpoint, while others are allowed to float or adjust freely to accommodate control of the set parameters. These are not necessarily scale specific. Due to the temperature sensitivity of XtractCF®, a typical approach to drying has been to control the inlet temperature, outlet temperature, drying gas flow rate, and atomization gas pressure/flow rate at setpoint values, while allowing the liquid feed rate to float and adjust to compensate.

### Example 4. Testing Various Extract Formulations and Drying Conditions

This example describes various extract formulations and drying conditions that were tested using different spray dryers.

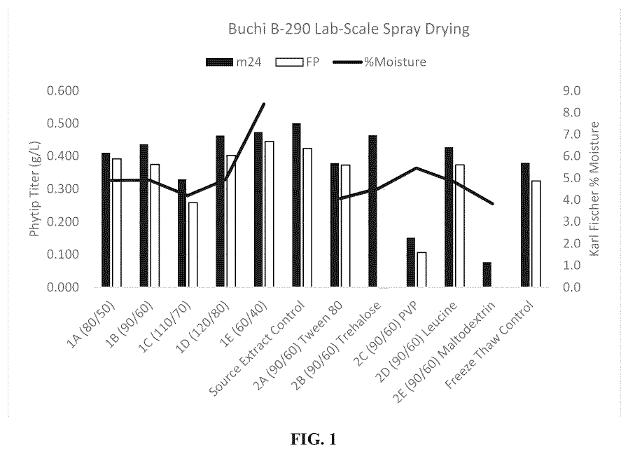

Formulation tests were conducted using a Buchi-290 lab-scale spray dryer to evaluate several common spray drying excipients as single component formulations. Spray dried powder samples were reconstituted into liquid form prior to testing XpressCF® performance. FIG. 1 shows initial titer and residual moisture results from XpressCF® (XCF) testing. In FIG. 1, respective inlet and outlet temperatures are noted in parenthesis for dried samples as (Inlet Temperature/Outlet Temperature). Samples 1A-1E are unformulated spray dried samples and samples 2A-2E are formulated spray dried samples with varying inlet temperature/outlet temperature as indicated. Protein A resin in a pipet tip, also referred herein as “Phytip,” is used in high throughput format on laboratory automation for rapid purification and titer quantification using A280 absorbance. Flower plate, also referred herein as “FP”, is a small multi-well plate without pH and DO control (˜1 ml reactions). Trehalose dihydrate (100 g/L, sample 2B) was a promising excipient, followed by leucine (10 g/L, sample 2D), and then Tween 80 (0.1%, sample 2A). Low initial titer results were obtained with PVP (100 g/L, sample 2C) and maltodextrin (100 g/L, sample 2E). Residual moisture was in the range of approximately 3-6% for the tested dried samples. Unformulated dried XtractCF ° showed good initial activity recovery. Control samples were unformulated liquid extract. There was some observed titer impact of freeze-thaw on controls.

Based on the results in FIG. 1, the 2-8° C. and room temperature stability were evaluated for dried XtractCF® formulated with 100 g/L trehalose dihydrate as well as unformulated. Testing included using multiple extract lots and drying conditions, as shown in Table 4.

TABLE 4
Buchi B-290 Lab-Spray Drying Stability

| | | | | | Titer | |
|---|---|---|---|---|---|---|
| | | | | | Normalized | |
| Timepoint | Storage | | Extract | Outlet | to Control | % |
| (months) | Condition | Formulation | Lot | T (° C.) | 100% | Moisture |
| Control | Control | None | DR-6-2 | n/a | 100 | n/a |
| Control | Control | None | DR 7-6 | n/a | 100 | n/a |
| Control | Control | None | DR 8-7 | n/a | 100 | n/a |
| Freeze-Thaw | Freeze-Thaw | None | DR 8-7 | n/a | 87 | n/a |
| Control | Control | | | | | |
| 8 Month | Room Temp | None | DR 7-6 | 40 | 0 | 7.59 |
| 8 Month | Room Temp | None | DR 8-7 | 40 | 1 | 8.42 |
| 8 Month | Room Temp | 100 g/L TDH | DR6-2 | 60 | 57 | 5.42 |
| 8 Month | 2-8° C. | None | DR 6-2 | 60 | 30 | 7.36 |
| 8 Month | 2-8° C. | None | DR 7-6 | 40 | 13 | 7.59 |
| 8 Month | 2-8° C. | None | DR 8-7 | 40 | 62 | 8.42 |
| 8 Month | 2-8° C. | 100 g/L TDH | DR 6-2 | 60 | 105 | 5.42 |
| 8 Month | 2-8° C. | 100 g/L TDH | DR 8-7 | 60 | 102 | 5.26 |
| 13 Month | Room Temp | 100 g/L TDH | DR 8-7 | 60 | 79 | 3.67 |
| 13 Month | Room Temp | 100 g/L TDH | DR 8-7 | 80 | 106 | 3.18 |
| 13 Month | 2-8° C. | None | DR 8-7 | 40 | 18 | 8.42 |
| 13 Month | 2-8° C. | None | DR 8-7 | 40 | 34 | 4.62 |
| 13 Month | 2-8° C. | None | DR 8-7 | 80 | 20 | 3.46 |
| 13 Month | 2-8° C. | None | DR 6-2 | 60 | 41 | 4.08 |
| 13 Month | 2-8° C. | None | DR 6-2 | 60 | 35 | 3.87 |
| 13 Month | 2-8° C. | 100 g/L TDH | DR 8-7 | 60 | 131 | 3.67 |
| 13 Month | 2-8° C. | 100 g/L TDH | DR 8-7 | 80 | 119 | 3.18 |
| 13 Month | 2-8° C. | 100 g/L TDH | DR 6-2 | 60 | 125 | 3.63 |
| 18 Month | Room Temp | 100 g/L TDH | DR 6-2 | 60 | 58 | 3.63 |
| 18 Month | 2-8° C. | 100 g/L TDH | DR 8-7 | 60 | 85 | 3.67 |
| 18 Month | 2-8° C. | 100 g/L TDH | DR 8-7 | 80 | 79 | 3.18 |

The samples in Table 4 were prepared and dried for both unformulated and formulated with 100 g/L trehalose. Drying was performed on a lab scale Buchi B-290 spray dryer. XCF testing used batch reactions in a Micro-24 microbioreactor with 30% XtractCF® to express product anti-CD74 antibody for the 8- and 13-month samples, and trastuzumab for the 18-month samples. Source liquid and dried XtractCF® was from either DR 6-2, DR7-6, and DR 8-7. Sample extract lots, residual moisture, and dryer outlet temperatures are noted for respective samples. As shown in Table 4, promising stability was observed for the 2-8° C. storage condition out to 18 months. Unformulated XtractCF® by comparison showed limited shelf-life stability indicating the importance of having a stabilizing formulation component present. Higher performance is observed for room temperature storage for the 100 g/L trehalose formulation compared with unformulated extract, even when stored at 2-8° C.

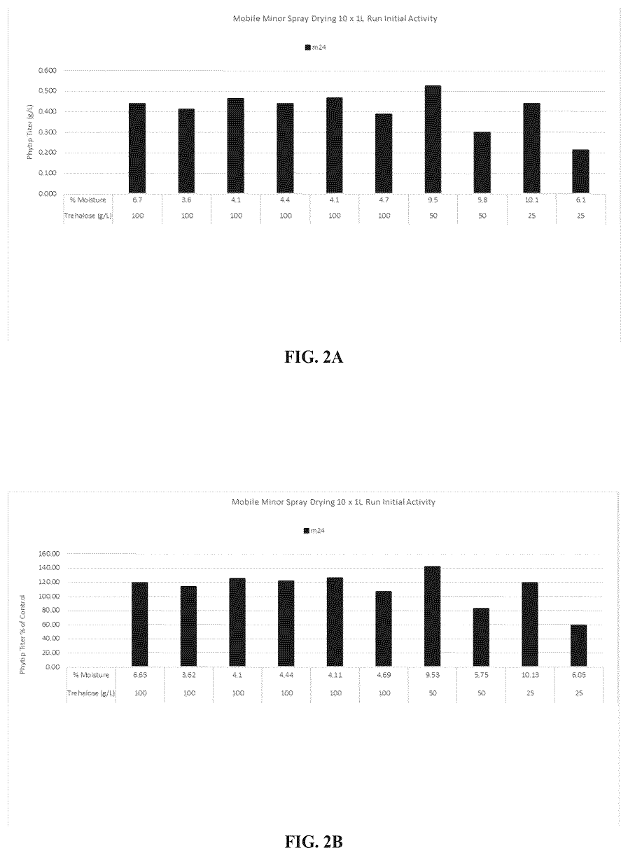

The drying process was scaled up to pilot scale drying on a Mobile Minor PSD-1. Several trehalose formulations were tested, including 100 g/L, 50 g/L, and 25 g/L, with some drying process condition scouting included as well. As shown in FIG. 2A and FIG. 2B, comparable initial activity was obtained at the three tested trehalose formulation levels. The moisture levels for the 50 g/L and 25 g/L formulations were observed to be higher than the 100 g/L formulation owning to the lower feedstock overall solids content which contributes to lower drying efficiency and less overall water evaporation as a result. The source unformulated liquid extract was used as a control.

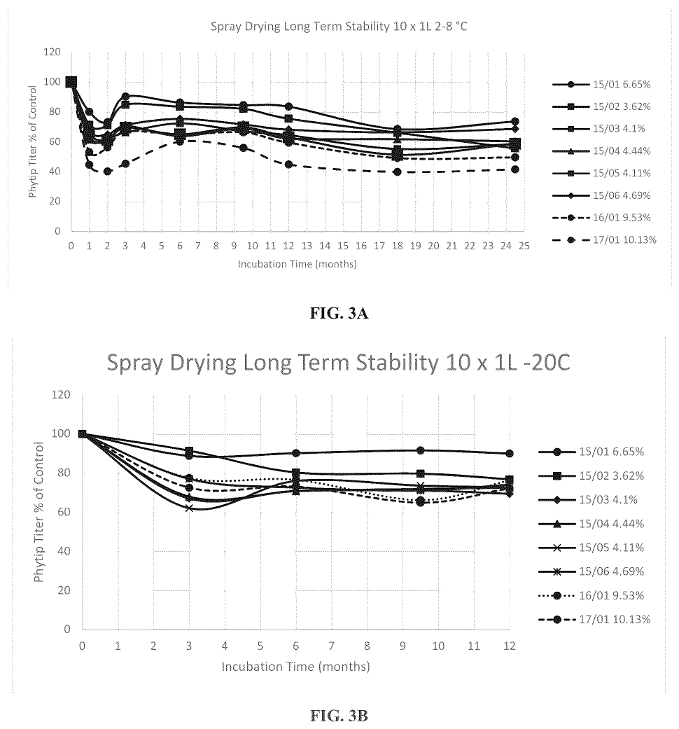

A subset of the samples from FIG. 2A and FIG. 2B with promising initial activity were tested in long term stability studies at storage conditions −20° C., 2-8° C., and room temperature. FIG. 3A and Table 5 show the stability results from the 2-8° C. storage condition. The 2-8° C. storage condition was evaluated out to 24.5 months. Calculated rates of titer loss are listed in Table 5.

TABLE 5
Titer for Certain Trehalose Formulations

| | | Residual Moisture | Rate of Titer Loss |
|---|---|---|---|
| Batch | Formulation | (%) | (% per month) |
| 15/XX | 100 g/L trehalose | 3.62-6.65 | 0.30-1.59 |
| | dihydrate | | |
| 16/01 | 50 g/L trehalose | 9.53 | 1.02 |
| | dihydrate | | |
| 17/01 | 25 g/L trehalose | 10.13 | 1.06 |
| | dihydrate | | |

As shown in Table 5, the rates for all TDH concentrations were in the range of 0.3 to 1.6% per month. However, the 50 g/L and 25 g/L formulations have higher moisture than 100 g/L, which confounds the ability to make a direct comparison purely based on the formulation concentration since the moisture level can impact stability. The −20° C. storage condition shows minimal titer loss evaluated over 12 months, as shown in FIG. 3B, compared with the 2-8° C. condition, and the room temperature condition shows significant activity losses over 8 months, including a clear distinction between levels of formulation, with higher levels of TDH being more stable, as shown in FIG. 3C. The control used in this testing was a t=0 reconstituted dried extract stored frozen (≤−65 ° C.) and was thawed upon need for use in testing.

The results presented above suggest trehalose is useful as a single component stabilizer. Therefore, 100 g/L trehalose was selected for further testing.

The minimum target drying feed flow rate was 10 kg/hr. The experiments were therefore repeated using a PSD-2 dryer that had been modified to achieve higher than the typically designed gas flow rates assessed to have potential to be able to meet desired throughputs. Although the drying chamber dimensions in comparison to the gas flow rate for a modified PSD-2 are from a technical perspective different than in a PSD-3, namely that the PSD-2 chamber is narrower than the PSD-3 chamber, the capacity for drying can be described as similar, since both the chamber size/dimensions and gas flow rates contribute to drying performance and efficiency.

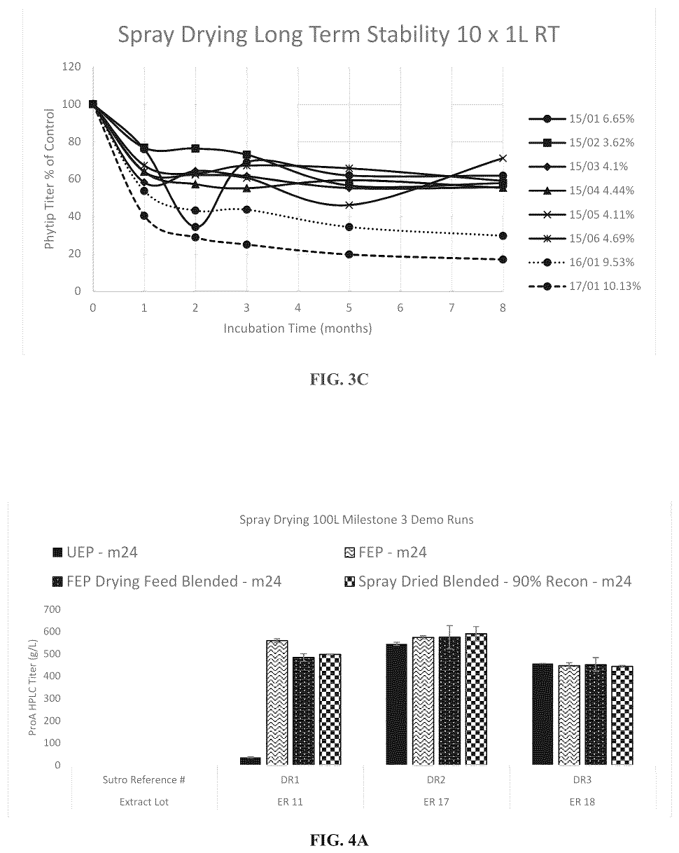

The testing involved initial process ranging of four conditions of 25 L each, from which a single process condition was selected. That condition was run for three successive runs of 100 L, each using a different extract lot to demonstrate process robustness. The results are shown in FIG. 4A and FIG. 4B. As shown in FIG. 4A and FIG. 4B, for each demonstration run, samples tested included unformulated extract pool (UEP), formulated extract pool (FEP), a blend of formulated liquid samples taken throughout each 100 L demo run (FEP Drying Feed Blended), and a blend of reconstituted dried XtractCF® samples taken throughout each 100 L demo run (Spray Dried Blended). Formulated samples comprised 100 g/L trehalose. All three runs demonstrating approximately 100% activity recovered for the respective dried blend compared with the formulated blend.

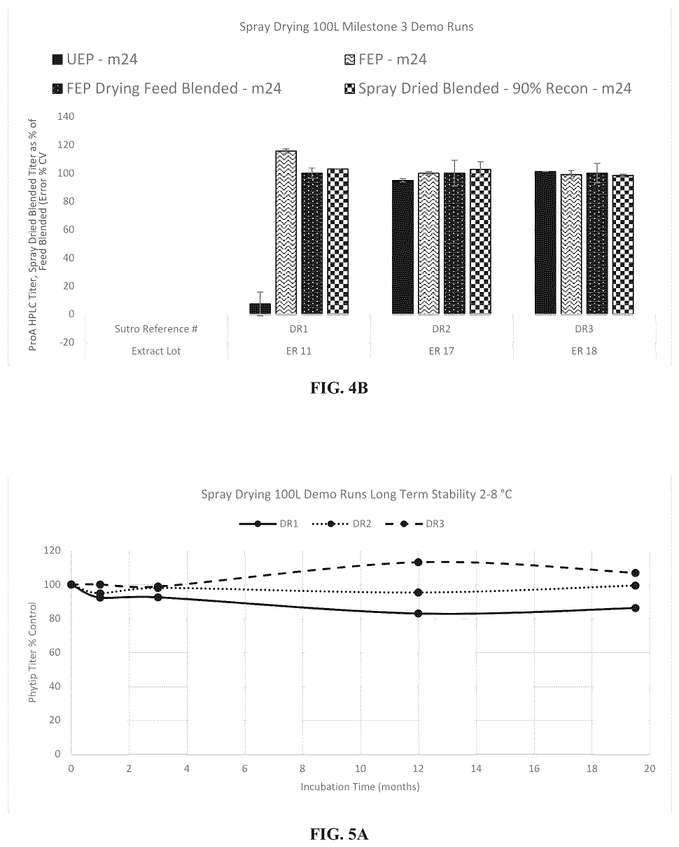

Materials from the demonstration runs were tested on long term stability. As shown in FIG. 5A, FIG. 5B, and FIG. 5C, the results show minimal if any drop in activity over approximately 19.5 months when the extracts were stored at −20° C. and 2-8° C., and a modest drop at approximately 19.5 months when the extracts were stored at room temperature (approximately 20° C.) to approximately 70-90% recovered activity. Controls used in testing were t=0 reconstituted dried extract.

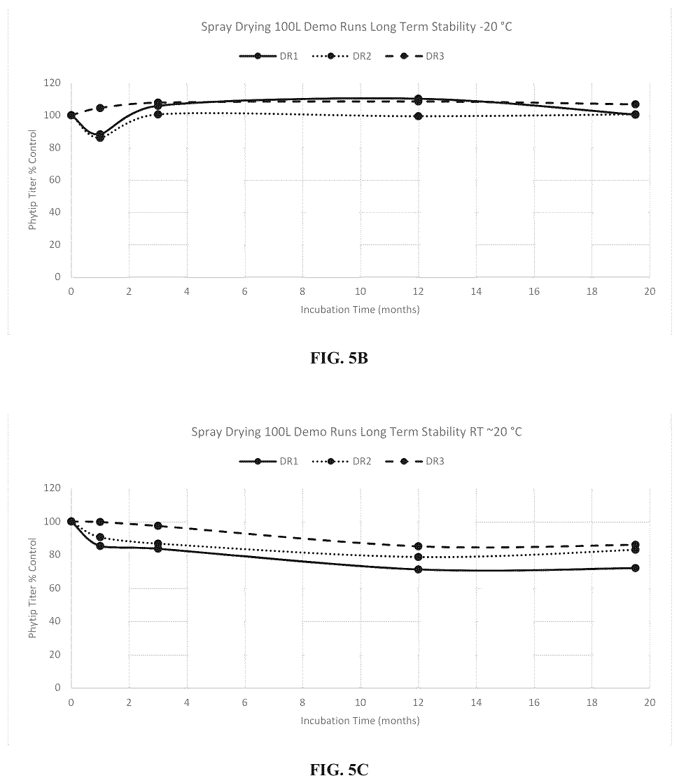

Select stability samples timepoints were tested in a scale-up stirred tank reactor configuration (DASbox, 200 ml XCF reaction volume), considered to be more representative of large-scale bioreactors compared with the Micro-24. DASbox, also referred herein as “DB,” is a small, stirred tank bioreactor system with pH and DO control (˜100-200 ml reactions). FIG. 6A and FIG. 6B show stability data at approximately 14 months demonstrating activity recovery of >90% when the extracts were stored at −20° C., approximately 80% when the extracts were stored at 2-8° C., and approximately 60-65% when the extracts were stored at room temperature. This testing utilized expression of product anti-folate receptor alpha antibody using pre-fabricated light chain (PFLC). Controls used in testing were t=0 reconstituted dried extract.

Additional formulation screening was conducted. The scope of testing included screening numerous single and multicomponent formulations including (1) sugars/polyols (2) amino acids (3) detergents/polyethylene glycol and (4) macromolecular crowding agents. Motivated by the above data that suggested possibly acceptable stability at 50 g/L trehalose compared with 100 g/L trehalose, an intermediate 75 g/kg trehalose formulation was evaluated here.

Table 6 shows a list of tested formulations, the corresponding XpressCF initial activity results, the reaction configuration tested (batch or fed-batch), and the expression product used. As shown in Table 6, the sample codes 1119, 0120, and 0220 represent November 2019, January 2020, and February 2020, respectively. XCF reaction configuration (batch or fed-batch) and expression product are noted for each tested formulation. All testing was performed with 37.5% extract by volume. Extract lots used were 19006-10 (November 2019), 19013-01 (January 2020), and 19011-04 (February 2020). m24, also known as micro-24, is a small multi-well microbioreactor system with pH and DO control (approximately 5 ml reactions). 19013-01 was a high bioburden lot suspected to contribute at least in part to lower than expected titers from January 2020 testing motivating additional testing in February 2020.

TABLE 6
Pilot-Scale Mobile Minor ® Spray Drying Compiled
Formulation Scouting Activity Results

| | | | Titer % of | | |
|---|---|---|---|---|---|
| | | m24 Avg | Respective | | |
| | | Titer | Batch or | | |
| Sample | Formulation | (g/L) | Fed Control | Reaction | Product |
| 0220A | TDH 100 g/L | 0.711 | 114 | Batch | anti-folate |
| | | | | | receptor alpha |
| | | | | | antibody PFLC |
| 0220K | TDH 75 g/kg | 0.717 | 114 | Batch | anti-folate |
| | | | | | receptor alpha |
| | | | | | antibody PFLC |
| 0220C | TDH 75 g/kg | 0.667 | 107 | Batch | anti-folate |
| | 2HPB- | | | | receptor alpha |
| | Cyclodextrin | | | | antibody PFLC |
| | 25 g/kg | | | | |
| 0220I | TDH 100 g/L | 0.668 | 107 | Batch | anti-folate |
| | | | | | receptor alpha |
| | | | | | antibody PFLC |
| 0220E | TDH 75 g/kg | 0.637 | 102 | Batch | anti-folate |
| | | | | | receptor alpha |
| | | | | | antibody PFLC |
| 0220E | TDH 75 g/kg | 0.591 | 101 | Batch | anti-CD74 |
| | | | | | antibody |
| Fed | Liquid Control | 0.899 | 100 | Fed | anti-folate |
| Control | (19011-04), Fed | | | | receptor alpha |
| | | | | | antibody PFLC |
| Fed | Liquid Control | 0.897 | 100 | Fed | anti-folate |
| Control | (19011-04), Fed | | | | receptor alpha |
| | | | | | antibody PFLC |
| Fed | Liquid Control | 0.645 | 100 | Fed | anti-CD74 |
| Control | (19013-01), Fed | | | | antibody |
| Fed | Liquid Control | 0.797 | 100 | Fed | anti-CD74 |
| Control | (19011-04), Fed | | | | antibody |
| Fed | Liquid Control | 0.776 | 100 | Fed | anti-CD74 |
| Control | (19011-04), Fed | | | | antibody |
| Fed | Liquid Control | 0.649 | 100 | Fed | anti-CD74 |
| Control | (19013-01), Fed | | | | antibody |
| Fed | Liquid Control | 0.386 | 100 | Fed | anti-CD74 |
| Control | (19006-10), Fed | | | | antibody |
| Fed | Liquid Control | 0.434 | 100 | Fed | anti-CD74 |
| Control | (19006-10), Fed | | | | antibody |
| Batch | Liquid Control | 0.296 | 100 | Batch | anti-CD74 |
| Control | (19006-10), | | | | antibody |
| | Batch | | | | |
| Batch | Liquid Control | 0.364 | 100 | Batch | anti-CD74 |
| Control | (19006-10), | | | | antibody |
| | Batch | | | | |
| Batch | Liquid Control | 0.452 | 100 | Batch | anti-CD74 |
| Control | (19013-01), | | | | antibody |
| | Batch | | | | |
| Batch | Liquid Control | 0.493 | 100 | Batch | anti-CD74 |
| Control | (19013-01), | | | | antibody |
| | Batch | | | | |
| Batch | Liquid Control | 0.587 | 100 | Batch | anti-CD74 |
| Control | (19011-04), | | | | antibody |
| | Batch | | | | |
| Batch | Liquid Control | 0.601 | 100 | Batch | anti-CD74 |
| Control | (19011-04), | | | | antibody |
| | Batch | | | | |
| Batch | Liquid Control | 0.622 | 100 | Batch | anti-folate |
| Control | (19011-04), | | | | receptor alpha |
| | Batch | | | | antibody PFLC |
| Batch | Liquid Control | 0.627 | 100 | Batch | anti-folate |
| Control | (19011-04), | | | | receptor alpha |
| | Batch | | | | antibody PFLC |
| 0220K | TDH 75 g/kg | 0.588 | 98 | Batch | anti-CD74 |
| | | | | | antibody |
| 0220I | TDH 100 g/L | 0.569 | 95 | Batch | anti-CD74 |
| | | | | | antibody |
| 0220A | TDH 100 g/L | 0.544 | 93 | Batch | anti-CD74 |
| | | | | | antibody |
| 0220C | TDH 75 g/kg | 0.530 | 90 | Batch | anti-CD74 |
| | 2HPB- | | | | antibody |
| | Cyclodextrin | | | | |
| | 25 g/kg | | | | |
| 0220D | LMH 100 g/kg | 0.779 | 87 | Fed | anti-folate |
| | | | | | receptor alpha |
| | | | | | antibody PFLC |
| 0220G | TDH 75 g/kg | 0.528 | 84 | Batch | anti-folate |
| | Amino Acid | | | | receptor alpha |
| | Select Mix 10 | | | | antibody PFLC |
| | g/kg | | | | |
| 0220J | LMH 100 g/kg | 0.699 | 78 | Fed | anti-folate |
| | | | | | receptor alpha |
| | | | | | antibody PFLC |
| 0220G | TDH 75 g/kg | 0.444 | 74 | Batch | anti-CD74 |
| | Amino Acid | | | | antibody |
| | Select Mix 10 | | | | |
| | g/kg | | | | |
| 1119-1 | TDH (100 g/L) | 0.264 | 68 | Fed | anti-CD74 |
| | | | | | antibody |
| 0220H | LMH 100 g/kg | 0.599 | 67 | Fed | anti-folate |
| | 2HPB- | | | | receptor alpha |
| | Cyclodextrin | | | | antibody PFLC |
| | 25 g/kg | | | | |
| 0220B | LMH 100 g/kg | 0.599 | 67 | Fed | anti-folate |
| | | | | | receptor alpha |
| | | | | | antibody PFLC |
| 0120J | LMH 100 g/L | 0.426 | 66 | Fed | anti-CD74 |
| | | | | | antibody |
| 0120K | LMH 100 g/L | 0.410 | 63 | Fed | anti-CD74 |
| | | | | | antibody |
| 0220J | LMH 100 g/kg | 0.489 | 61 | Fed | anti-CD74 |
| | | | | | antibody |
| 0120B | Raffinose 90 g/L | 0.395 | 61 | Fed | anti-CD74 |
| | | | | | antibody |
| 0220D | LMH 100 g/kg | 0.475 | 61 | Fed | anti-CD74 |
| | | | | | antibody |
| 0120F | Raffinose 90 g/L | 0.386 | 60 | Fed | anti-CD74 |
| | | | | | antibody |
| 0120I | LMH 100 g/L | 0.355 | 55 | Fed | anti-CD74 |
| | | | | | antibody |
| 0220B | LMH 100 g/kg | 0.423 | 55 | Fed | anti-CD74 |
| | | | | | antibody |
| 0220F | LMH 100 g/kg | 0.481 | 54 | Fed | anti-folate |
| | Amino Acid | | | | receptor alpha |
| | Select Mix 5 g/kg | | | | antibody PFLC |
| 0120D | Raffinose 90 g/L | 0.322 | 50 | Fed | anti-CD74 |
| | | | | | antibody |
| 0220H | LMH 100 g/kg | 0.398 | 50 | Fed | anti-CD74 |
| | 2HPB- | | | | antibody |
| | Cyclodextrin | | | | |
| | 25 g/kg | | | | |
| 0120H | Raffinose 90 g/L + | 0.324 | 50 | Fed | anti-CD74 |
| | PEG200 15 g/L | | | | antibody |
| 0220L | LMH 100 g/kg | 0.445 | 50 | Fed | anti-folate |
| | Amino Acid | | | | receptor alpha |
| | Select Mix 10 | | | | antibody PFLC |
| | g/kg | | | | |
| 0220F | LMH 100 g/kg | 0.364 | 47 | Fed | anti-CD74 |
| | Amino Acid | | | | antibody |
| | Select Mix 5 g/kg | | | | |
| 1119-8 | PMA Amino | 0.202 | 47 | Fed | anti-CD74 |
| | Acid Mix (13.03 | | | | antibody |
| | g/L) | | | | |
| 0120M | TDH 100 g/L + | 0.285 | 44 | Fed | anti-CD74 |
| | PEG200 5 g/L | | | | antibody |
| 0120L | LMH 100 g/L + | 0.266 | 41 | Fed | anti-CD74 |
| | PEG200 15 g/L | | | | antibody |
| 0120E | TDH 100 g/L + | 0.258 | 40 | Fed | anti-CD74 |
| | PEG200 30 g/L | | | | antibody |
| 0120A | TDH 100 g/L | 0.249 | 39 | Fed | anti-CD74 |
| | | | | | antibody |
| 0220L | LMH 100 g/kg | 0.305 | 38 | Fed | anti-CD74 |
| | Amino Acid | | | | antibody |
| | Select Mix 10 | | | | |
| | g/kg | | | | |
| 0120C | TDH 100 g/L + | 0.224 | 35 | Fed | anti-CD74 |
| | PEG200 15 g/L | | | | antibody |
| 0120G | TDH 100 g/L + | 0.217 | 33 | Fed | anti-CD74 |
| | Leucine 8 g/L | | | | antibody |
| 1119-7 | Leucine (8 g/L) | 0.089 | 20 | Fed | anti-CD74 |
| | | | | | antibody |
| 1119-5 | Mannitol (50 g/L) | 0.063 | 16 | Fed | anti-CD74 |
| | | | | | antibody |
| 1119-2 | Mannitol (100 | 0.030 | 8 | Fed | anti-CD74 |
| | g/L) | | | | antibody |
| 1119- | Mannitol (100 | 0.028 | 6 | Fed | anti-CD74 |
| 10 | g/L), Sucrose (15 | | | | antibody |
| | g/L), Sorbitol (15 | | | | |
| | g/L) | | | | |
| 1119-9 | Mannitol (80 g/L) | 0.021 | 5 | Fed | anti-CD74 |
| | | | | | antibody |
| 1119-6 | Mannitol (100 | 0.013 | 3 | Fed | anti-CD74 |
| | g/L), PMA | | | | antibody |
| | Amino Acid Mix | | | | |
| | (13.03 g/L) | | | | |
| 1119-3 | Mannitol (100 | 0.013 | 3 | Fed | anti-CD74 |
| | g/L), Leucine (8 | | | | antibody |
| | g/L) | | | | |
| 1119- | Mannitol (100 | 0.013 | 3 | Fed | anti-CD74 |
| 11 | g/L), PEG200 (15 | | | | antibody |
| | g/L), PMA | | | | |
| | Amino Acid Mix | | | | |
| | (13.03 g/L) | | | | |
| 1119-4 | Mannitol (100 | 0.003 | 1 | Fed | anti-CD74 |
| | g/L), PEG200 (15 | | | | antibody |
| | g/L), Leucine (8 | | | | |
| | g/L) | | | | |
| 1119- | Mannitol (100 | 0.003 | 1 | Fed | anti-CD74 |
| 12 | g/L), PEG200 (15 | | | | antibody |
| | g/L) | | | | |

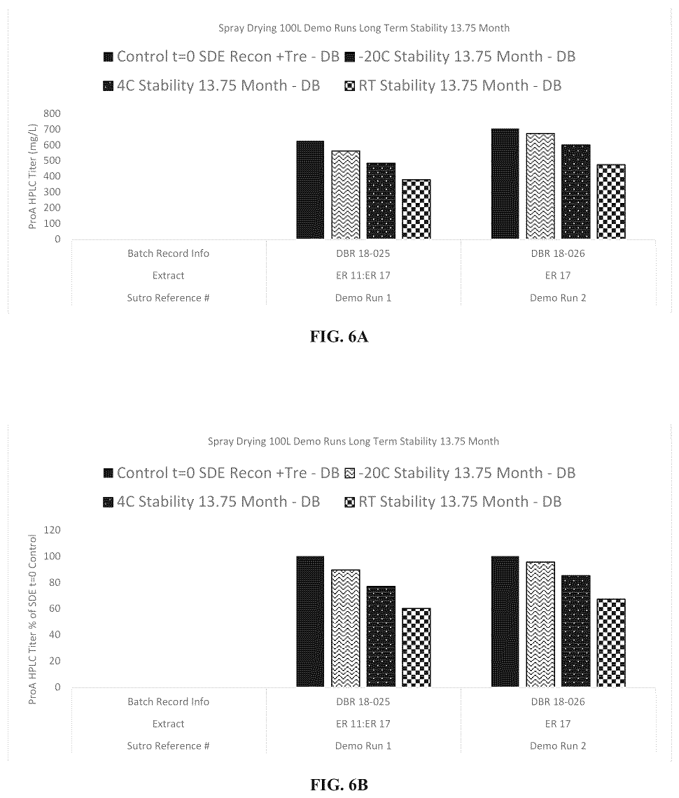

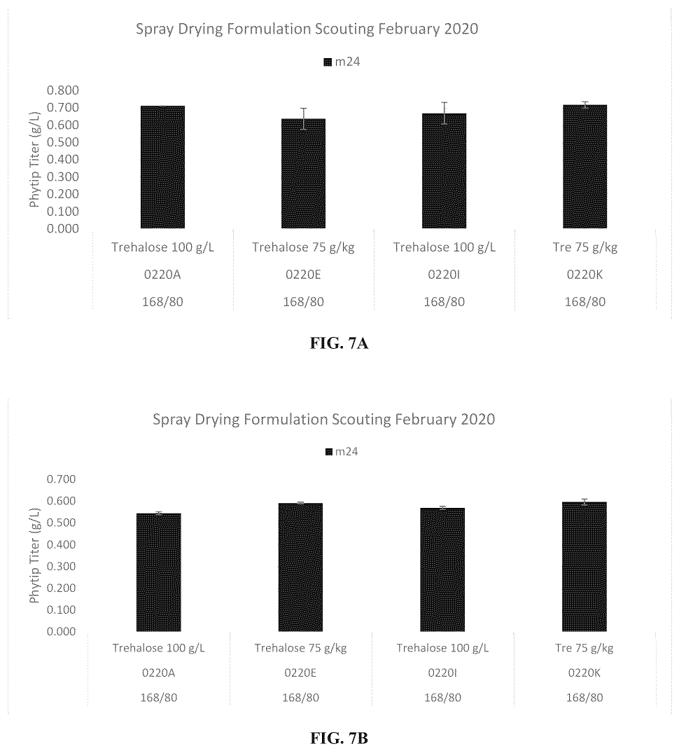

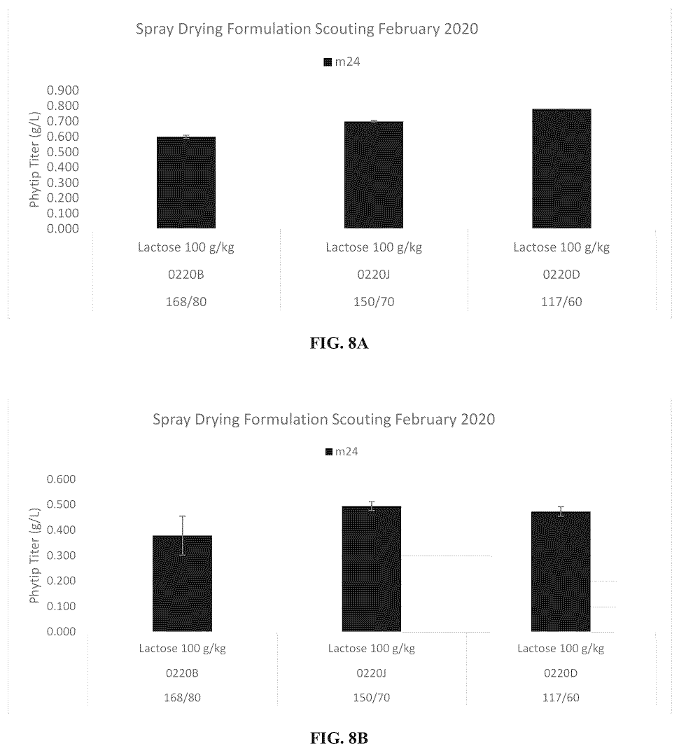

The data in Table 6 show a positive correlation between excipient glass transition temperature (T_{g}) and post-drying recovered activity. The top candidate formulations were single components (A) trehalose dihydrate 100 g/L, (B) trehalose dihydrate 75 g/kg, and (C) lactose monohydrate 100 g/kg, as shown in FIG. 7A, FIG. 7B, FIG. 8A, and FIG. 8B, which show promising results for the expression of two different antibody products. Initial activity with 100 g/kg lactose was more favorable for the 60° C. and 70° C. outlet temperatures compared with 80° C., so only these two conditions were pursued further. These single component formulations were tested on long term stability and show similar rates of titer loss as shown in FIG. 9 and Table 7. Some combination formulations containing either the high T_{g}/nonpolar/uncharged amino acid mix or 2-hydroxypropyl-β-cyclodextrin showed promising initial activity, but none offered any improvement over the TDH or LMH single component options.

TABLE 7
Trehalose Dihydrate & Lactose Monohydrate
Single Component Formulated XtractCF ® 2-
8° C. Long Term Stability.

| | | Residual Moisture | Rate Titer Loss |
|---|---|---|---|
| Batch | Formulation | (%) | (%/month) |
| 0220A | 100 g/L trehalose dihydrate | 2.23 | 2.15 |
| 0220I | 100 g/L trehalose dihydrate | 2.36 | 5.89 |
| 0220E | 75 g/kg trehalose dihydrate | 2.03 | 4.77 |
| 0220K | 75 g/kg trehalose dihydrate | 2.38 | 4.54 |
| 0220D | 100 g/kg lactose | 4.02 | 3.27 |
| | monohydrate | | |
| 0220J | 100 g/kg lactose | 3.55 | 3.40 |
| | monohydrate | | |

### PSD-3 (Sutro)

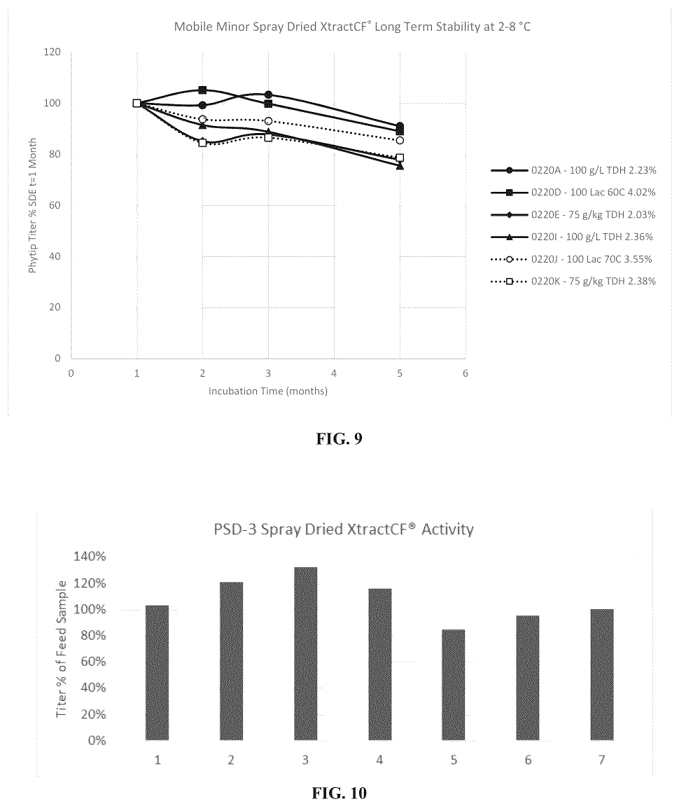

A series of ranging runs were conducted during the startup of the PSD-3 dryer. A Water Run was done first to test drying process operational ranges. Following that were several iterations of testing with extract to develop an understanding on process parameters ranges and outputs. Process and analytical data from these runs were used to select GMP production run conditions. The 75 g/kg TDH formulation was used in GEA PSD-3 drying runs and produced SDE with high activity recovery, as shown in FIG. 10.

In summary, the spray-dried bacterial extracts described in this example show high percentage activity and improved stability during storage with a variety of formulations. Such extracts have a longer self-life at room temperature, 2-8° C., and −20° C. compared to control extracts, such as additive-free (unformulated) extracts. The results of the above studies show that the additive containing spray-dried bacterial extracts increase the stability of bacterial extracts in CFP S reactions.

All publications, issued patents, and patent applications cited in this specification are herein incorporated by reference as if each individual publication or patent application were specifically and individually indicated to be incorporated by reference.

1. A method for producing a stable, spray-dried bacterial extract for cell-free protein synthesis, the method comprising:
i. combining a bacterial extract comprising lysed bacterial components with a composition comprising trehalose, lactose, leucine, or raffinose to yield a mixture, wherein the bacterial extract is able to synthesize a target protein from a template nucleic acid encoding the target protein in a cell-free protein synthesis reaction; and
ii. spray-drying the mixture to produce the stable, spray-dried bacterial extract.

2. The method of claim 1, wherein the bacterial extract comprising lysed bacterial components is a liquid or rehydrated bacterial extract.

3. The method of claim 1, wherein the composition comprises trehalose or lactose.

4. The method of claim 1, wherein the mixture comprises about 25 to 200 g/kg trehalose.

5. The method of claim 4, wherein the mixture comprises about 50 to 100 g/kg trehalose.

6-48. (canceled)

49. A spray-dried bacterial extract for cell-free protein synthesis comprising:
dried, lysed bacterial components; and
a composition comprising trehalose, lactose, leucine, or raffinose,
wherein upon rehydration, the extract is able to synthesize a target protein from a template nucleic acid encoding the target protein.

50. The spray-dried extract of claim 49, wherein the composition comprises trehalose or lactose.

51. The spray-dried extract of claim 49, wherein the composition comprises about 25 to 200 g/kg of trehalose.

52. The spray-dried extract of claim 49, wherein the composition comprises about 50 to 100 g/kg of trehalose.

53. The spray-dried extract of claim 51, wherein the trehalose is trehalose dihydrate (TDH).

54-92. (canceled)

93. A method of preparing a spray-dried extract, comprising the steps of:
(i) providing a liquid bacterial extract comprising components for cell-free synthesis of a target protein from a template nucleic acid encoding the target protein;
(ii) producing droplets of the liquid bacterial extract;
(iii) contacting the droplets with a gas to evaporate liquid from the droplets;
(iv) separating the dried extract from the gas and smaller particles; and
(v) collecting the spray-dried extract.

94. The method of claim 93, wherein prior to step (i) the liquid bacterial extract is sterile filtered.

95. The method of claim 94, wherein the sterile filtered liquid bacterial extract is activated by heating.

96. The method of claim 95, wherein a composition comprising trehalose, lactose, leucine, or raffinose is added to the activated sterile filtered liquid bacterial extract prior to step (ii).

97. method of claim 96, wherein the composition comprises about 25 to 200 g/kg trehalose, about 25 to 200 g/kg lactose, about 5 to 10 g/L leucine, or about 25 to 200 g/L raffinose.

98-116. (canceled)

117. A spray-dried extract for cell-free protein synthesis, the spray dried extract comprising:
dried, lysed bacterial components; and
one or more of a stabilizer,
wherein the stabilizer has a glass transition temperature (Tg) of at least about 90° C., and
wherein the concentration of the stabilizer is between about 5 g/L and 200 g/L or between about 25 g/kg and 200 g/kg in the liquid extract prior to spray-drying.

118. The spray-dried extract of claim 117, wherein the stabilizer is selected from trehalose, lactose, and leucine.

119. The spray-dried extract of claim 118, wherein the stabilizer comprises about 25 to 200 g/kg trehalose, about 25 to 200 g/kg lactose or about 5 to 10 g/L of leucine.

120. The spray-dried extract of claim 119, wherein the trehalose is trehalose dihydrate (TDH) and the lactose is lactose monohydrate (LMH).

121-124. (canceled)

125. A method for producing a target protein from a spray dried extract, the method comprising:
reconstituting the spray dried extract of claim 117;
providing a template nucleic acid encoding the target protein; and
generating the target protein.
